(19) United States
(12) Patent Application Publication
(10) Pub. No.: US 2024/0371496 A1
(43) Pub. Date: Nov. 7, 2024

(54) IMPLEMENTING REAL-TIME MONITORING AND EVALUATION

(71) Applicant: Excellent Capacity Limited, HONG KONG (HK)

(72) Inventor: Hong WANG, Cupertino, CA (US)

(21) Appl. No.: 18/651,256

(22) Filed: Apr. 30, 2024

Related U.S. Application Data
(60) Provisional application No. 63/464,491, filed on May 5, 2023.

(51) Int. Cl. G16H 20/70; A61B 5/00; G16H 10/60; G16H 50/20

(52) U.S. Cl. CPC G16H 20/70; A61B 5/0002; G16H 10/60; G16H 50/20

(57) ABSTRACT

The present disclosure describes techniques for implementing real-time data monitoring and evaluation. Each of a plurality of client systems is associated with a particular user. A personalized database corresponding to each particular user is generated based on data received from each of the plurality of client systems. The personalized database comprises information indicating frequencies of a plurality types of events associated with the particular user, an environmental condition corresponding to each event, behavioral characteristics of the particular user at each event, a timestamp of each event, a length of time of each event, and biological and biochemical information of the particular user. A condition of the particular user is determined based on the personalized database. A personalized recommendation is generated and presented to the particular user for managing the condition.

### CROSS-REFERENCE TO RELATED APPLICATION

This disclosure claims priority to U.S. Provisional Patent Application No. 63/464,491, filed on May 5, 2023, which is incorporated herein by reference in its entirety.

### BACKGROUND

Companies and/or individuals are increasingly utilizing and/or producing a large quantity of data. Such data may need to be monitored and/or analyzed. Improved techniques for data monitoring and/or analysis to improve quality of life are desirable.

### BRIEF DESCRIPTION OF THE DRAWINGS

The following detailed description may be better understood when read in conjunction with the appended drawings. For the purposes of illustration, there are shown in the drawings example embodiments of various aspects of the disclosure; however, the invention is not limited to the specific methods and instrumentalities disclosed.

FIG. 1 shows an example system for implementing real-time monitoring and evaluation in accordance with the present disclosure.

FIG. 2 shows an example system for implementing real-time monitoring and evaluation in accordance with the present disclosure.

FIG. 3 is a schematic diagram illustrating an example cloud-based server that may be used in accordance with the present disclosure.

FIG. 4 shows an example client system in accordance with the present disclosure.

FIG. 5 shows an example wearable device in accordance with the present disclosure.

FIG. 6 shows an example server-side process for implementing real-time monitoring and evaluation in accordance with the present disclosure.

FIG. 7 shows an example server-side process for implementing real-time monitoring and evaluation in accordance with the present disclosure.

FIG. 8 shows an example process for training a machine learning model in accordance with the present disclosure.

FIG. 9 shows an example process for encrypting a database in accordance with the present disclosure.

FIG. 10 shows an example client-side process for implementing real-time monitoring and evaluation in accordance with the present disclosure.

FIG. 11 shows an example process for uploading data in accordance with the present disclosure.

FIG. 12 shows an example computing device which may be used to perform any of the techniques disclosed herein.

### DETAILED DESCRIPTION OF ILLUSTRATIVE EMBODIMENTS

Some medical conditions, such as dementia (e.g., Alzheimer's disease (AD), Parkinson's disease (PD)) have long incubation periods, such as periods of several years or even decades. Early-stage detection of these medical conditions is of great significance. An individual diagnosed with early stage of dementia, for example, may still be able to function independently (e.g., drive, work, be part of social activities, etc.) despite having some mild symptoms, such as memory lapses (e.g., forgetting familiar words or the location of everyday objects). Early-stage detection can avoid worsening of the condition. Early-stage detection allows affected individuals to receive timely treatment and avoid worsening of the symptoms and/or condition. If an individual is not diagnosed until the condition has progressed to an advanced stage, treatment will be more difficult, and some symptoms may never be relieved. Early-stage detection can therefore improve the quality of life of affected individuals, as affected individuals can participate in drug treatment, exercise therapy, physical therapy, etc. to reduce various symptoms, such as muscle stiffness, tremors, and difficulty in moving. Early-stage detection can also significantly help with scientific search, such as by providing scientists with more research samples to study the pathogenesis of these conditions and to provide a better basis for future treatments.

However, early-stage detection of these conditions is difficult. Early symptoms of these medical conditions are often ignored or attributed to other conditions and/or normal aging. Further, many of these medical conditions do not have a cure. No drugs currently exist to cure dementia, for example. Thus, by the time individuals are diagnosed with these medical conditions, they have often been suffering from an irreversible disease for many years. As such, improved techniques for early-stage detection of the medical conditions are needed.

Described herein are devices, systems, and methods for early-stage detection of the conditions, such as dementia, cancers, and diabetes. The techniques described herein rely on the real time and on-site monitoring and evaluation of data collected from a large quantity (e.g., hundreds, thousands, millions, etc.) of client systems, including users' wearable devices, and devices surrounding users in their daily activities. The collected data may indicate the behaviors and corresponding changes in the daily activities and biological and biochemical information of the respective users of the client systems. The large quantity of client systems can upload detected data to a remote cloud platform. The cloud platform can utilize artificial intelligence (AI) algorithm technology, including AI big data and big models (or large models, large-scale AI models), such as language big models, visual big models, visual language big models, transformer-based big models (e.g., GPT (Generative Pre-Trained Transformer)), BERT (Bidirectional Encoder Representations from Transformers), CNNs (convolutional neural networks) big model, RNNs (Recurrent neural networks) big model, GNNs (Generative Adversarial Networks) big model, RL (Reinforcement Learning) big model, graph neural network big models, super alignment big models, pre-trained big models, multimodal big models that one of way is to include at least two of those big models mentioned above, etc.) analysis technology to accurately classify individuals into two groups: one group that is affected by a medical condition, and a second group that is normal (e.g., not affected by the medical condition), as one of application scenarios. The cloud platform can utilize artificial intelligence (AI) algorithm technology and big data and big model analysis technology to accurately classify individuals into sub-groups based on the user's condition and severity. These classifications can significantly improve detection sensitivity, specificity and accuracy, and provide more effective medical solutions for patients to classify disease stages, track medical efficacy, and develop new drugs.

Data can be collected from various client systems associated with a single user to monitor a single user's behavior and to detect events, such as changes behavior and corresponding changes in his or her daily life and/or changes in his or her biological and biochemical information. If an event reaches a threshold level, this can indicate that the user has a high probability of having a disease, such as an early stage disease. Additionally, or alternatively, data can be collected from a large quantity (e.g., hundreds, thousands, millions, etc.) of client systems associated with a plurality of different users, such as hundreds, thousands, or millions of users, to compare commonalities and characteristics among all of the users from the plurality of users. The commonalities and characteristics among all of the users from the plurality of users can be compared to statistically determine events dependent of the users' different characteristics, such as different ages, sexes, races, profession, education backgrounds, personal medical historys, family historys, living habits, time frames with epidemic case, regions with special environmental condition, drugs and other medical treatments, etc. Determining the events dependent of the users' different characteristics can be used to identify possible root causes of the events, and in turn, to trigger better classification of users' medical conditions, to improve detection sensitivity, specificity and accuracy, to more effectively screen diseases, especially early-stage diseases, and to more efficiently develop drugs and other medical treatments.

The cloud platform can issue warnings to individuals before the obvious and severe symptoms of AD, PD, cancers, and diabetes patients appear and are discovered. The data collected and analyzed by the cloud platform can be provided to medical staff with objective and timely personalized information regarding the behaviors and symptoms of early-stage patients. The data collected and analyzed by the cloud platform can be used for treatment tracking feedback and medical drug development and treatment to reduce the number of patients suffering from the medical conditions, reduce the suffering of the patients, and reduce the medical and social costs caused by the medical conditions.

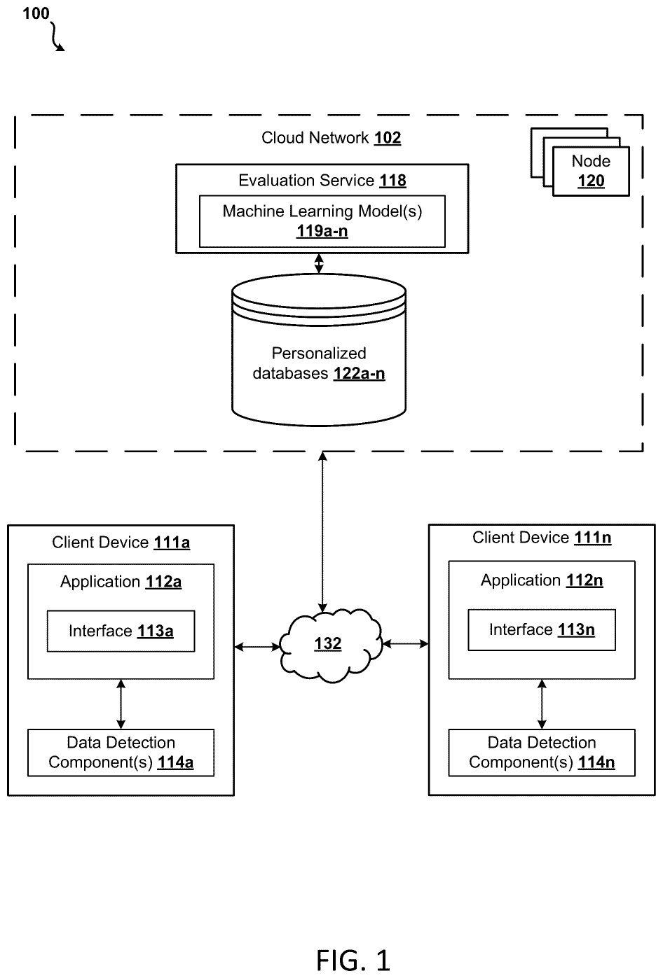

FIG. 1 illustrates an example system 100 for implementing real-time monitoring and evaluation. The system 100 can include a cloud network 102 and a plurality of client systems 111a-n. The cloud network 102 and plurality of client systems 111a-n (collectively client system 111) may communicate with each other via one or more networks 132.

The cloud network 102 may be located at a data center, such as a single premise, or be distributed throughout different geographic locations (e.g., at several premises). The cloud network 102 may provide the services via the one or more networks 132. The network 132 comprise a variety of network devices, such as routers, switches, multiplexers, hubs, modems, bridges, repeaters, firewalls, proxy devices, and/or the like. The network 132 may comprise physical links, such as coaxial cable links, twisted pair cable links, fiber optic links, a combination thereof, and/or the like. The network 132 may comprise wireless links, such as cellular links, satellite links, NearLink, Wi-Fi links, Bluetooth link, and/or the like.

The cloud network 102 may comprise a plurality of computing nodes 120 that host a variety of services. In an embodiment, the nodes 120 host an evaluation service 118. The evaluation service 118 may be configured to distribute (e.g., transmit, send) data (e.g., information) to the plurality of client systems 111a-n via a variety of transmission techniques. The data distributed by the evaluation service 118 may comprise video, audio, textual data, a combination thereof, and/or the like. In an embodiment, the data may be distributed to the plurality of client systems 111a-n via the network 132. The data may be streamed to the plurality of client systems 111a-n. In an embodiment, the plurality of client systems 111a-n may each comprise an application 112a-n. The application 112a-n can output (e.g., display, render, present) the data to a user associated with the client system 111a-n.

Each of the plurality of client systems 111a-n may comprise a wearable device, such as a head-mounted eyepiece, eye-glasses, hat-mounted devices, necklace hanging devices, chest-worn devices that are hung on the chest, devices that are hung on the back, and devices that are hung on the shoulders, limbs (including but not limited to fingers, wrists, arms, legs, feet, torso, neck, etc.), and devices that are hung on human or animal bodies that are exposed to the body surface and are moved due to daily activities of the body parts, which are not likely to damage the physical location and working status of the instruments and devices. Each of the plurality of client systems 111a-n may comprise any other type of computing device, such as a mobile device, a tablet device, laptop, a desktop computer, or other smart device (e.g., smart watch, smart speaker, smart glasses, smart helmet), a gaming device, a set top box, digital streaming device, robot, and/or the like.

The plurality of client systems 111a-n may be associated with one or more users. A single user may use one or more of the plurality of client systems 111a-n to access the cloud network 102. The users may be registered as users of the evaluation service (or application scenarios) 118 and may be users of the application 112a-n operating on the plurality of client systems 111a-n. The plurality of client systems 111a-n may stay in one location and/or travel to a variety of locations and use different networks to access the cloud network 102. Each of the plurality of client systems 111a-n may be mobile or stationary. Each of the plurality of client systems 111a-n may be positioned indoors, outdoors, in cars, on ships or in cabins, on drones (e.g., micro drones), and in or on aircraft, etc., so that users are surrounded by the plurality of client systems 111a-n almost all the time and at all locations in their daily activities. In this way, with the multimodal big model method, users are able to interact with the plurality of client systems 111a-n, and in turn interact with the cloud AI algorithm based local and/or remote expert consultant center with personalized databases.

The plurality of client systems 111a-n can collect and/or monitor data, such as in real-time. The data collected and/or monitored by a particular one of the client systems 111a-n may comprise information indicating the behaviors and corresponding changes in the daily activities of a user of that client system and/or biological and biochemical information of the user. The plurality of client systems 111a-n may comprise one or more data detection components 114a-n. The data detection component(s) 114a-n may be components (e.g., sensors, etc.) configured to collect and/or monitor the data, such as in real-time. The data detection component(s) 114a-n may comprise a visual component. The visual component may be configured to capture images and video (moving images) and perform visual recognition. The data detection component(s) 114a-n may comprise a hearing component. The hearing component may be configured to record audio signals and perform speech recognition. The data detection component(s) 114a-n may comprise an optical component. The optical component may be configured to inject incident light and detect the biological and biochemical information of a user.

In embodiments, the plurality of client systems 111a-n can detect changes to the behaviors in the daily activities and/or changes to the biological and biochemical information of the corresponding user over time. The plurality of client systems 111a-n can detect changes to the behaviors in the daily activities and/or changes to the biological and biochemical information of the corresponding user based on (e.g., using the collected and/or monitored data). The plurality of client systems 111a-n can generate a personalized database corresponding to the particular user based on the detected changes. The personalized database can be stored locally (e.g., on the client system), and/or stored remotely (e.g., on the cloud platform and artificial intelligence (AI) algorithm-based database). The personalized database can include information indicating frequencies of a plurality types of events associated with the particular user. The personalized database can include information indicating an environmental condition corresponding to each event. The personalized database can include information indicating behavioral characteristics of the particular user at each event. The personalized database can include information indicating a timestamp of each event. The personalized database can include information indicating a length of time of each event. The plurality of client systems 111a-n can detect changes to the behavior in the daily activities and/or changes to the biological and biochemical information of the corresponding user based on (e.g., using) the data in the personalized database.

In embodiments, the plurality of client systems 111a-n can determine a condition (e.g., a medical condition) of the particular user based on the data stored in the personalized database. For example, the plurality of client systems 111a-n can determine if the user has an early-stage medical condition, such as early-stage dementia. The plurality of client systems 111a-n can generate a personalized recommendation based on the condition, such as the early-stage medical condition. The plurality of client systems 111a-n can present information indicating the personalized recommendation to the user for managing the condition. The plurality of client systems 111a-n can present the information indicating the personalized recommendation to the user for managing the condition via an interface 113a-n of the application 112a-n.

In embodiments, the data collected and/or monitored by the plurality of client systems 111a-n can be sent to (e.g., uploaded to, directly uploaded to) to the evaluation service 118 (e.g., through wired or wireless transmission technology). The plurality of client systems 111a-n can be interconnected with the evaluation service 118. Artificial intelligence algorithms can be operated and/or stored in a database of the evaluation service 118.

In embodiments, the evaluation service 118 can receive the data collected and/or monitored by a particular one of the plurality of client systems 111a-n. The evaluation service 118 can receive the data from the plurality of client systems 111a-n. The evaluation service 118 may be configured to receive data from the plurality of client systems 111a-n in real-time (e.g., as it is collected by the plurality of client systems 111a-n). The evaluation service 118 can monitor the data (e.g., in real-time).

In embodiments, the evaluation service 118 can generate a plurality of personalized databases 122a-n. The evaluation service 118 can generate the plurality of personalized databases 122a-n based on (e.g., in response to) receiving the data from the plurality of client systems 111a-n. Each of the plurality of databases 122a-n can correspond to a particular user. The database 122a-n corresponding to a particular user may store the data received from the corresponding one of the plurality of client systems 111a-n. The personalized database 122a-n corresponding to a particular user may store information indicating frequencies of a plurality types of events associated with the particular user. The personalized database 122a-n corresponding to a particular user may store information indicating an environmental condition corresponding to each event. The personalized database 122a-n corresponding to a particular user may store information indicating behavioral characteristics of the particular user at each event. The personalized database 122a-n corresponding to a particular user may store information indicating a timestamp of each event. The personalized database 122a-n corresponding to a particular user may store information indicating and a length of time each event. The evaluation service 118 can encrypt the personalized databases 122a-n to prevent unauthorized access.

In embodiments, the evaluation service 118 can perform pattern recognition and/or determine a condition of a particular user based on the corresponding personalized database 122a-n. The evaluation service 118 can perform pattern recognition and/or determine a condition of a particular user based on (e.g., using) one or more machine learning models 119a-n. The evaluation service 118 can generate a personalized recommendation based on the condition. For example, the evaluation service 118 can generate a real-time notification of blood sugar test results for a diabetic patient. If the test result exceeds the preset blood sugar concentration warning limit, the user can be warned (e.g., via mobile phone, email, etc.). The evaluation service 118 can transmit (e.g., send) information indicating the personalized recommendation to one of the client systems associated with the particular user for display of the personalized recommendation, such as display of the personalized recommendation via one of the interfaces 113a-n.

In embodiments, the evaluation service 118 can compare the data stored in all of, or a subset of, the personalized databases 122a-n to compare commonalities and characteristics among a plurality of users. The commonalities and characteristics among all of the users from the plurality of users can be compared to statistically determine events dependent of the users' different characteristics, such as different ages, sexes, races, professions, education backgrounds, personal medical historys, family historys, living habits, time frames with epidemic case, regions with special environmental condition, drugs and other medical treatments, etc. Determining the events dependent of the users' different characteristics can be used to identify possible root causes of the events, and in turn, to trigger better classification of users' medical conditions, to improve detection sensitivity, specificity and accuracy, to more effectively screen diseases, especially early-stage diseases, and to more efficiently develop drugs and other medical treatments.

The plurality of computing nodes 120 may process tasks associated with the evaluation service 118. The plurality of computing nodes 120 may be implemented as one or more computing devices, one or more processors, one or more virtual computing instances, a combination thereof, and/or the like. The plurality of computing nodes 120 may be implemented by one or more computing devices. The one or more computing devices may comprise virtualized computing instances. The virtualized computing instances may comprise a virtual machine, such as an emulation of a computer system, operating system, server, and/or the like. A virtual machine may be loaded by a computing device based on a virtual image and/or other data defining specific software (e.g., operating systems, specialized applications, servers) for emulation. Different virtual machines may be loaded and/or terminated on the one or more computing devices as the demand for different types of processing services changes. A hypervisor may be implemented to manage the use of different virtual machines on the same computing device.

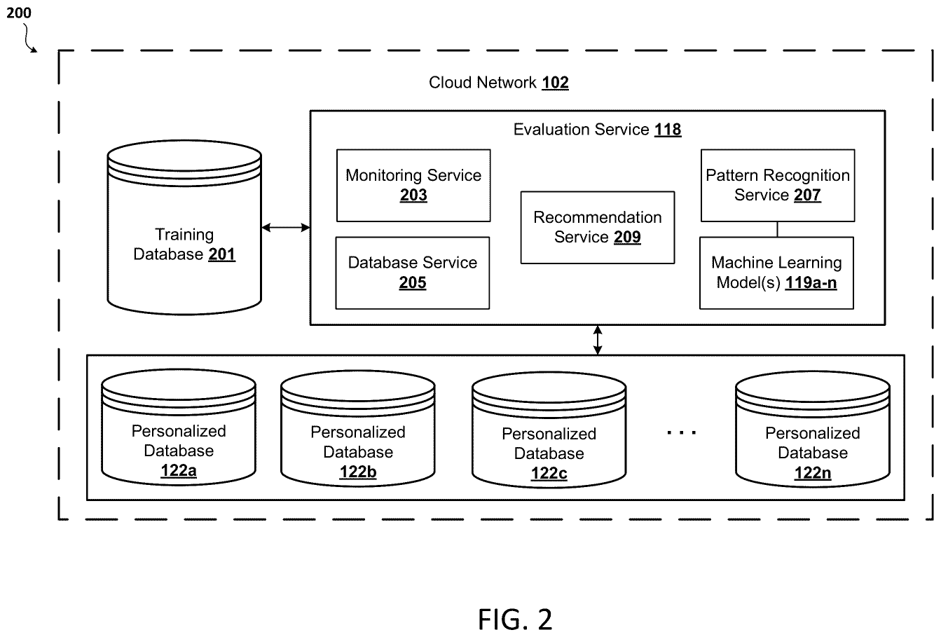

FIG. 2 illustrates an exemplary cloud network 102 in more detail. As described above, the cloud network 102 can include the evaluation service 118 and the plurality of personalized databases 122a-n. The evaluation service 118 can include a monitoring service 203, a database service 205, a recommendation service 209, a pattern recognition service 207, and the machine learning model(s) 119a-n.

In embodiments, the monitoring service 203 can receive the data collected and/or monitored by a particular the plurality of client systems 111a-n. The monitoring service 203 can receive the data from the plurality of client systems 111a-n. The monitoring service 203 can receive the data from the plurality of client systems 111a-n in real-time (e.g., as it is collected by the plurality of client systems 111a-n). The monitoring service 203 can monitor the data (e.g., in real-time, on-site).

In embodiments, the database service 205 can generate the plurality of personalized databases 122a-n. The database service 205 can generate the plurality of personalized databases 122a-n based on (e.g., in response to) receiving the data from the plurality of client systems 111a-n. Each of the plurality of personalized databases 122a-n can correspond to a particular user. For example, the personalized database 122a can correspond to a first user, the personalized database 122b can correspond to a second user, the personalized database 122c can correspond to a third user, and so on. The database service 205 may store the data received from the client system(s) corresponding to the first user in the personalized database 122a may store, the data received from the client system(s) corresponding to the second user in the personalized database 122b, the data received from the client system(s) corresponding to the third user in the personalized database 122c, and so on. The data stored in each of the plurality of personalized databases 122a-n can include information indicating frequencies of a plurality types of events associated with the corresponding user, information indicating an environmental condition corresponding to each event, information indicating behavioral characteristics of the corresponding user at each event, information indicating a timestamp of each event, and/or information indicating and a length of time each event. The database service 205 may encrypt the personalized databases 122a-n to prevent unauthorized access.

In embodiments, the pattern recognition service 207 can perform pattern recognition and/or determine a condition of a particular user based on the corresponding personalized database 122a-n. The pattern recognition service 207 can perform pattern recognition and/or determine a condition of a particular user based on (e.g., using) the machine learning models 119a-n. The recommendation service 209 can generate a personalized recommendation based on the condition. The recommendation service 209 can transmit (e.g., send) information indicating the personalized recommendation to one of the client systems associated with the particular user for display of the personalized recommendation.

The big data and big models based machine learning models 119a-n can include any machine learning or deep learning algorithm, such as one or more applied mathematical technology models. The applied mathematical technology models can include probability statistics and/or stochastic processes models, linear algebra and/or matrix theory models, methods or algorithms of tensors and tensor networks, calculus and numerical optimization models, group theory models, information theory models, optimization theory models, formal logic models, etc.

The big data and big models based machine learning models 119a-n can include one or more classification models (e.g., supervised learning algorithms for modeling or predicting discrete random variables), such as Naive Bayes (NB), K-Nearest Neighbors (KNN), Decision Tree (DT), Artificial Neural Network (ANN), Support Vector Machines (SVM), Random Forest (RF), Convolutional Neural Networks (CNN), Partial least squares discriminant analysis (PLS-DA), etc. The machine learning models 119a-n can include one or more regression models (e.g., supervised learning algorithm for predicting and modeling numerical continuous random variables), such as Partial Least Squares Regression (PLSR), Principal Component Regression (PCR), Mixed Logistic Regression (MLR), Support Vector Machine Regression (Support Vector Machines Regression (SVMR), Gradient Boosting Decision Tree (GBDT), Logistic Regression (LR), etc.

The big data and big models based machine learning models 119a-n can include one or more cluster analysis models (e.g., unsupervised machine learning technique that involves grouping data points and finding natural groups (i.e. clusters) of observed samples based on the internal structure of the data), such as k-means Clustering (k-MC), Mean Shift Clustering (MSC), and Density-based Spatial Clustering of Applications with Noise (DBSCAN), Expectation-Maximum (EM) Clustering with Gaussian Mixture Models (GMM-EM), Agglomerative Hierarchical Clustering (AHC), Graph Community Detection (GCD), Hierarchical Clustering Analysis (HCA), etc. The machine learning models 119a-n can include one or more dimensionality reduction models (e.g., models that, through some mathematical transformation, transform an original high-dimensional attribute space into a low-dimensional subspace), such as Principal Components Analysis (PCA), Linear Discriminant Analysis (LDA), Locally Linear Embedding (LLE), Laplacian eigenmaps. (Laplacian Eigenmaps, LE), and Partial Least Square Regression (PLS), etc.

To apply one or more of the big data and big models based machine learning models 119a-n, the algorithm model may need to be iterated based on new data. Model iteration methods can include, but are not limited to, online incremental learning methods (e.g., directly applying newly added data to further update the original model, the model is learned online, and machine learning obtains an original algorithm model that further amplifies the new data), methods of merging data (e.g., merging the algorithm training data that has been completed in stages and the newly added data, the model learns all the data offline, and machine learning obtains a new algorithm model), model fusion methods (e.g., using the prediction results of the original model as newly added features, and training a new algorithm model on the new data), and/or the like.

In embodiments, the database service 205 can generate a training database 201. The training database 201 can include (e.g., store) training data for training the machine learning models 119a-n. The database service 205 can generate the training data. To generate the training data, the database service 205 can aggregate information from the plurality of personalized databases 122a-n. The database service 205 can classify the plurality of users into a plurality of groups based on the aggregated information. The database service 205 can define new training data based on (e.g., using) the classifications. The database service 205 can store the new training data in the training database 201. The evaluation service 118 can train the machine learning models 119a-n on the new training data to improve an accuracy of condition prediction.

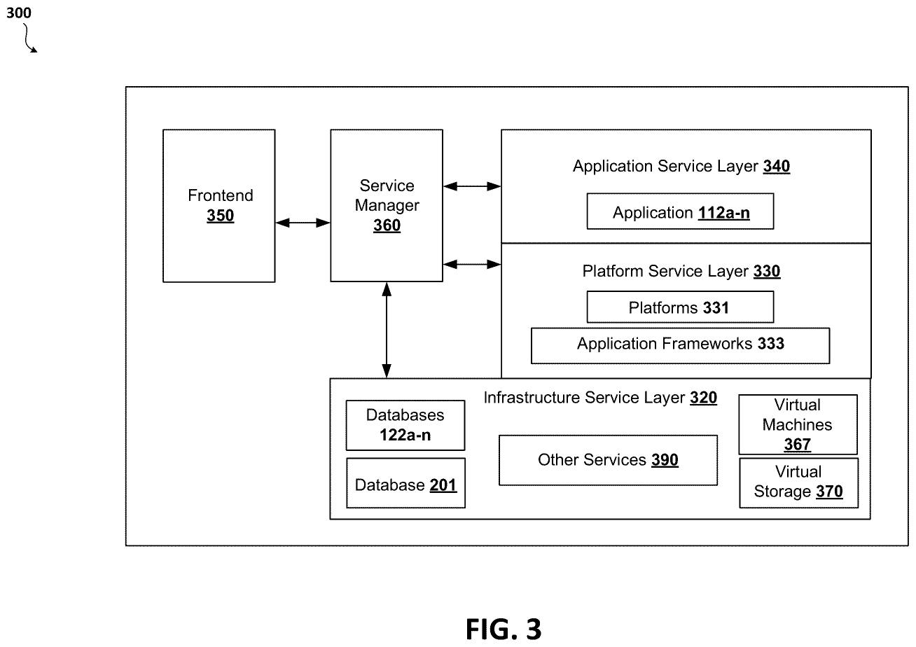

FIG. 3 is a schematic diagram illustrating an example cloud-based server 300 that may be used in accordance with the present disclosure. The cloud-based server 300 may provide infrastructure services, platform services, and software application services. The infrastructure services may include virtualized resources, such as virtual machines 367, virtual storage 370, and so on. The infrastructure services may also include virtualized services, such as database services and other services 390. Each of these infrastructure services may be deployed in an infrastructure service layer 320.

The scale and various aspects, such as data, connectivity, and dependency relationships within and between service components, of an infrastructure service deployment are configurable by an administrator user. For instance, the administrator user may submit a configuration specification to the cloud-based server 300 via a frontend interface 350 and service manager 360. The configuration specification can be translated into infrastructure and kernel level application programming interface (APIs) calls that create, re-create, move, or delete components such as virtual machines 367 and services, and assign or change attributes of the components.

In addition to the infrastructure services, the cloud-based server 300 may also provide platform services 331, such as an environment for running virtual machines 367 or a framework for developing and launching a particular type of software applications. The platform services 331 may be implemented in a platform service layer 330 over the infrastructure service layer 320, and may employ one or more infrastructure services configured in a particular manner. Configuration of platform services can be accomplished by program code written according to the APIs of the platform services and, optionally, the APIs of the infrastructure services that are employed in enabling the platform services.

In some examples, the cloud-based server 300 may also provide software application services in an application service layer 340. The application 112a-n can be installed on one or more virtual machines 367 or deployed in an application framework 333 in the platform service layer 330. The software application can also communicate with one or more infrastructure service components, such as databases 122a-n and/or database 201, in the infrastructure layer 320. The installation and configuration of the software application in the application service layer 340 can be accomplished through APIs of the software itself and the APIs of the underlying platform and infrastructure service components.

Depending on the type of services, a cloud-service user may be granted different levels of control in configuring the services. For example, if a software application service is employed, an administrator user is given control over how the software application is configured. If a platform service is employed, an administrative user is given control over how the platform and/or application frameworks are configured. Similarly, if infrastructure services are employed, an administrative user is given control over the particular infrastructure services employed.

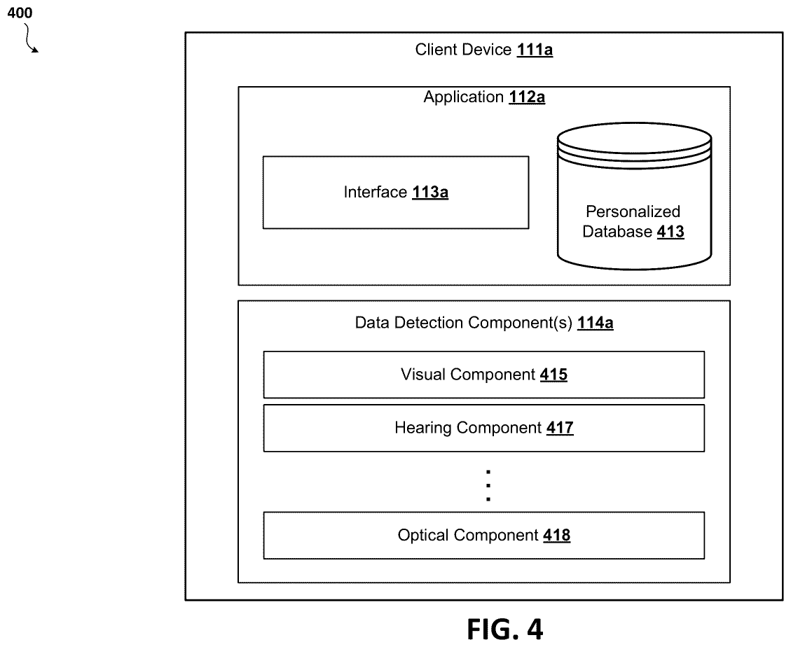

FIG. 4 illustrates an exemplary client system 111a in more detail. As described above with regard to FIG. 1, the client system 111a can collect and/or monitor data, such as in real-time. The data collected and/or monitored by the client system 111a may comprise information indicating the behaviors and corresponding changes in the daily activities of a user of that client system and/or biological and biochemical information of the user. The client system 111a may comprise one or more data detection components 114a-n. The data detection component(s) 114a-n may be components (e.g., sensors, etc.) configured to collect and/or monitor the data, such as in real-time.

The data detection component(s) 114a-n may comprise a visual component 415. The visual component 415 may be configured to capture images and video (moving images) and perform visual recognition. The visual component 415 may comprise a camera or a video camera, artificial intelligence automatic vision function (including but not limited to camera photography, video shooting, etc.), visual recognition function, including but not limited to image recognition, video recognition, face recognition, facial expression recognition, human eye iris recognition, human body form recognition, object recognition, animal and plant recognition, biometric recognition, and/or other functions.

The data detection component(s) 114a-n may comprise a hearing component 417. The hearing component 417 may be configured to record audio signals and perform speech recognition. The hearing component 417 can be configured to perform computer artificial intelligence automatic hearing (including but not limited to recording, etc.) functions, which can automatically start recording and identify audio signals, including but not limited to speech recognition, language recognition and other functions of the tracked target person. The hearing component 417 can include a computer artificial intelligence automatic hearing component that is equipped with an automatic auditory language automatic translation function.

The data detection component(s) 114a-n may comprise one or more sensors configured to perform artificial intelligence perception and positioning of the movement of the instruments and devices body (including but not limited to the limbs, head and neck, waist, etc. of the wearer wearing the instruments and devices of the invention) over time and space. For example, the data detection component(s) 114a-n may comprise accelerometers, gyroscopes, levels, inertial navigation systems (INS), Global Positioning System (GPS) receivers, and GPS, a Beidou Navigation Satellite System, and/or any other positioning systems connected with positioning function instruments and devices with horizontal X-Y coordinate positioning and longitudinal (e.g., direction perpendicular to the horizontal direction) Z-directional calibration.

In embodiments, the client system 111a can detect changes to behaviors in the daily activities and/or changes to the biological and biochemical information of the corresponding user over time. The client system 111a can detect changes to behaviors in the daily activities and/or changes to the biological and biochemical information of the corresponding user based on (e.g., using) the collected and/or monitored data. The client system 111a can generate a personalized database 413 corresponding to the particular user based on the detected changes. The personalized database 413 can be stored locally (e.g., on the client system 11a), and/or stored remotely (e.g., on the cloud platform and AI algorithm based database). The personalized database 413 can include information indicating frequencies of a plurality types of events associated with the particular user. The personalized database 413 can include information indicating an environmental condition corresponding to each event. The personalized database 413 can include information indicating behavioral characteristics of the particular user at each event. The personalized database 413 can include information indicating a timestamp of each event. The personalized database 413 can include information indicating a length of time of each event. The client system 111a can detect changes to the behavior in a user's daily activities and/or changes to the biological and biochemical information of the corresponding user based on (e.g., using) the data in the personalized database.

In embodiments, the client system 111a can detect changes to the behaviors in a user's daily activities and/or changes to the biological and biochemical information of the user over time. The client system 111a can detect changes to the behaviors in a user's behaviors and corresponding changes in the daily activities and/or changes to the biological and biochemical information of the user based on (e.g., using) the collected and/or monitored data. For example, the client system 111a can detect basic data of the daily behavior assessment of the target person or animal tracked or monitored by the client system 111a as the data baseline, to detect the degree of change over time that is different from the baseline, including but not limited to the frequency of events of the monitored person, the environmental conditions of the scene where it occurs, the behavioral characteristics of the person when it occurs, the time point of each occurrence, the length of time and the severity of the incident, etc., to build a personalized database of people.

The client system 111a can detect impairments in the memory of the user (e.g., short-term memory disorders) for events that occurred recently, including but not limited to the inability to pronounce previously familiar words, phrases, names of people, etc. during speech, difficulty in using words, etc. For example, the client system 111a can detect if it is difficult for a user to accurately describe the specific content of the previous meals, if it is difficult to describe the events that the user performed or experienced within a few hours, even a few minutes, etc.

The client system 111a can detect changes in speech speed, language delay, and unnecessary language repetition of the character's language, including but not limited to asking the same questions repeatedly, inappropriate use of even wrong words to replace the correct words used in the person's normal language, and meaningless speech, hallucinatory speech, abnormal suspicious speech that is almost borderline vulgar, boring, or even slander without any basis, deceptive language, accidental or intentional misrepresentation of facts and distorting reality (e.g., referring to a deer as a horse), and/or the like.

The client system 111a can detect changes in the cognitive function of the user. For example, the client system 111a can detect a user's behavior that abnormal delusions and doubts about events, forgetfulness of understanding of other people, etc. The client system 111a can assess the cognitive function of the user via cognitive test and/or a neurological status test (e.g., Mini-Mental State Examination (MMSE), Montreal Cognitive Assessment (MoCA), General Practitioner Cognitive Assessment Scale (GPCOG), Memory Impairment Screening Scale (MIS), Quick Cognitive Screening Scale for the Elderly (QCSS-E), Simple Cognitive Screening Questionnaire for the Elderly (BECSI), Memory and Executive Screening Scale (MES), Mild Cognitive Impairment Screening Scale (sMCI), Cambridge Neuropsychological Automated Test Battery (CANTAB), Unified Parkinson's disease rating scale score, non-motor symptom scale score, the cognitive assessment test for Parkinson's disease research (CAB-PK), etc.). The client system 111a can assess the cognitive function of the user using epidemiological data, family genetic information associated with the user, medical history of the user, and family medical history, and/or the like.

The client system 111a can detect changes in behavior of the user. The client system 111a can detect abnormal behavior, including but not limited to abnormal indifference, acrimony, and unfounded suspicion towards others and things, wearing shoes backwards without knowing it, putting electronic equipment (e.g., mobile phones, watches, headphones, etc.), glasses and other items that do not need to be refrigerated or heated are placed in refrigerators, microwave, ovens, and other equipment, mixed things, lying on the ground for no reason to sleep, etc.

The client system 111a can detect abnormal behavioral movement of the user. For example, the client system 111a can detect meaningless delays in regular movements, uncoordinated and coherent actions when dressing, non-purposeful wandering, slowness or inability to avoid obstacles when encountering and seeing them while traveling, or even due to forgetfulness, suddenly crying for no reason, suddenly attacking others unintentionally or intentionally, refusing to accept care from others, and/or the like.

The client system 111a can detect changes in eyes or facial expressions of the user. For example, the client system 111a can detect short-term defocusing, trance, sluggishness, decreased blinking, rapid eye movement, sleep behavior disorder, etc. The client system 111a can detect if a facial expression of the user indicates that the user is in a short-term daze, trance or sluggish state. The client system 111a can detect olfactory dysfunction, including olfactory detection threshold, odor identification and odor recognition ability decay over time, etc. The client system 111a can detect changes in a cognition of the user. For example, the client system 111a can detect cognitive confusion, including but not limited to anxiety, suspicion, suspicion, resentment, etc. The client system 111a can detect a decreased attention of the user, such as an inability to plan things, and inability to handle complex matters. The client system 111a can detect if the user is having difficulty completing tasks that the user was previously able to accomplish. The client system 111a can detect if the user has a problem with the sense of space, for example, when a visual function of the user is normal and the user is looking for something, the object is right in front of him or her but the user cannot recognize the object (e.g., so called riding a donkey looking for the donkey). The client system 111a can detect if the user demonstrates an increasing difficulty or even inability to learn new things.

The client system 111a can detect changes in a user's behavior and attitude toward other individuals and things: for example, from paying attention to the one's own personal care (including but not limited to daily personal hygiene, dressing up, etc.) to gradually paying less attention to or even ignoring personal care; from being selfless and generous, always caring about others and even self-denial and benefit to others, indifferent, not caring about others, narrow-minded, depressed, hesitant, etc., to long-term decreased interest in favorite activities and hobbies (such as special sports, artistic TV or online programs, chess and cards activities, or human-machine games, etc.)

The client system 111a can detect changes in diet, such as recently added foods rich in metallic aluminum for a long time, including but not limited to foods rich in alum (potassium aluminum (potassium aluminum sulfate), sodium alum (sodium aluminum sulfate), ammonium alum (aluminum ammonium sulfate)), potassium aluminum chloride, etc.) foods, including but not limited to fried dough sticks, fried cakes, fried shrimp crackers, jellyfish, porous and soft pastry snacks, pasta with added baking powder containing alum, etc. The client system 111a can detect changes in the daily tableware used by the users, both historically and/or recently increased items containing aluminum metal, including but not limited to the inner pot of a high-pressure rice cooker, frying pans, rice cookers, bowls, plates, spoons, soup spoons, chopsticks, baking pans for grilled food, aluminum foil for grilled food, water kettles, thermos cups, thermos cups, tea sets, etc.

The client system 111a can detect changes in behavior and movement, such as trembling, stiffness of limbs, slow movement, unstable posture, etc. The client system 111a can detect tremors (e.g., involuntary abnormal movements of one or more parts of the body, including but not limited to limbs, shoulders, head, neck, or the muscles of these areas). These involuntary and abnormal movements encompass single, indirect repetitive, and repetitive dyskinesias, such as static tremors, shaking, twitching, sliding (including but not limited to non-subjective voluntary stretching, lifting, and sweeping actions, occurring once or intermittently more than once, in body parts such as arms), or involuntary finger tapping (for example, often associated with hand movements resembling ‘rolling pills’ where the index finger involuntarily approaches the thumb, causing the two fingers to intertwine, resembling the action of a pharmacist making pills). The client system 111a can detect limb stiffness, involuntary twitching of limbs or fingers, slowness of movement and hypokinesia: patients in the early stages of PD will initially have difficulty performing fine movements of daily living (such as writing, sewing, or dressing up), unstable posture or even falling, abnormal gait when walking, including but not limited to small broken steps, unbalanced gait, staggering, one arm bending involuntarily when walking and having difficulty or even no ability to swing, a user losing balance, and/or a user falling, especially backward falling.

The client system 111a can detect changes in the writing of a user, such as fonts become smaller and smaller over time. The client system 111a can detect difficult moving, difficulty in eating, manifested as difficulty in swallowing, which may occur periodically over time, variable clumsiness, and inflexibility in washing, dressing and other fine movements, etc. The client system 111a can detect changes in hearing of a user, changes in the stool quality of a user, decreased concentration.

The data detection component(s) 114a-n may comprise an optical component 418. The optical component 418 may be configured to inject incident light and detect the biological and biochemical information of a user. The optical component 418 may be configured to detect the blood glucose concentration in a user's body fluid (e.g., saliva, urine, tears, subcutaneous body fluids, etc.). The optical component 418 can include spectroscopic probes and/or fiber optical probes (referred to as detection probes) and/or a probe window that incidents and collects light. The light signal that can be collected from samples is a certain part of a user's body, including but not limited to the user's fingers, wrists, forearms, arms, legs, feet, waist and abdomen, trunk, neck, deep inside nasal cavity, etc., to non-invasively or minimal invasively detect the blood glucose concentration in subcutaneous body fluids and capillaries close to the epidermis at the user body area that closes or even touches the detection probe window; or use a wearable instrument and device to make incident light irradiate on the tears on the surface of the user's eyes to detect the blood sugar (or blood glucose) concentration in the tears. Regular noninvasive monitoring of the user's blood sugar can be arranged, for example, every 1 hour or even 3 or 5 minutes a time. The time span can cover the time period of user's daily activities such as eating, sleeping, working, and exercising, etc. After necessary data processing (including but not limited to data fitting, noise reduction, etc.), the client system 111a can wirelessly upload the detection data to the receiving end, including but not limited to personal mobile phones, computers (including tablets, PADs, etc.), wearable devices wireless display equipment, detection system database center, remote expert system. Once the real-time detection data contains the measured blood glucose value exceeding the warning limit threshold, the detection system center and remote expert system activate the alarm system, including but not limited to mobile phone text messages, mobile phone alarm bells or vibrations, emails, etc., to notify the character being tested and his or her relative, guardian or emergency contact person.

Using an optical detection probe to detect the biomarkers of cancers from user's body fluids and/or tissues: The data detection component(s) 114a-n may comprise an optical component 418. The optical component 418 may be configured to inject incident light and detect the biological and biochemical information of a user. The optical component 418 may be configured to detect the biomarkers of cancers in a user's body fluid (including but not limited to blood, serum, saliva, urine, sweat, tears, cerebrospinal fluid (CSF), pleural effusion, subcutaneous body fluids (including but not limited to subcutaneous blood, interstitial fluids, lymph, etc.), tissue fluids, semen, gynecological secretions, etc., etc.) and/or tissues. The optical component 418 can include spectroscopic probes or fiber optical probes (referred to as detection probes) and/or a probe window that incidents and collects light. The light signal that can be collected from samples containing biological and biochemical information from user's body fluids and/or tissues, to non-invasively or minimal invasively detect the biomarkers of cancers in user's body fluids and/or tissues including subcutaneous body fluids and capillaries close to the epidermis; or use a wearable instrument and device to make incident light irradiate on the tears on the surface of the user's eyes to detect the biomarkers of cancers in the tears. After necessary data processing (including but not limited to data fitting, noise reduction, etc.), the client system 111a can wirelessly or by wire upload the detection data to the receiving end, including but not limited to personal mobile phones, computers (including tablets, PADs, etc.), wearable devices wireless display equipment, detection system database center, remote expert system. The detection data can be collected from user A, user B, . . . user N, etc., all those data can be uploaded to the cloud and AI algorithm based database to carry out data analysis, to classify cancer related user group and health group.

The biomarkers of cancers include but not limited to genes, proteins and molecular markers related to cancer (including but not limited to DNA, Z-DNA, T-DNA, circulating tumor DNA (ctDNA), cfDNA, cfDNA methylation and its abnormalities, circulating tumor cells (CTC), RNA, mRNA, miRNA, micro-RNA, Exosomes (Exosomes, extracellular vesicles (EVs)), CEA, AFP, PSA (TPSA, FPSA, etc.), NSE, SCC, CA-125, CA15-3, CA19-9, CA72-4, CA242, A-549, CD20, CD30, CD34, CD45, CD99, EMA, hCG, s100, SF, TPA, TPA, ALK, BRAF, GFAP, EGFR, ER, KRAS, MET, p53, TP53, HER-2, hC, Mucin-1, PC3, LPCaP, HSP90-α, etc.), amino acids (including but not limited to cysteine, etc.), branched-chain amino acids and amino acid variations, etc., Adenosine Triphosphate (ATP), Ferritin, atomic groups or molecular groups (including but not limited to groups containing phosphorus and sulfur), carotenoides, β-carotene, lycopene, lutein (Lutein), zeaxanthin (Zeaxanthin), glycosaminoglycans (GAGome), red blood cells (RBCs), platelets, low carbon alkanes, polycyclic aromatic hydrocarbons, etc.

The client system 111a can detect biomarkers of diseases, including dementia (including Alzheimer's disease (AD), Parkinson disease (PD), etc.), cancers, diabetes, contained in human body fluids and tissues. Human body fluids can include but are not limited to blood, saliva, urine, sweat, tears, cerebrospinal fluid (CSF), pleural effusion, subcutaneous body fluids (including but not limited to subcutaneous blood, interstitial fluids, lymph, etc.), tissue fluids, semen, gynecological secretions, etc. Human tissues include but are not limited to the hippocampus, substantia nigra area, pineal gland in the brain, skin, etc. The client system 111a can detect biomarkers using minimally invasive or non-invasive optical spectroscopy's spectral fingerprint (or bar code), etc.), cancer spectral fingerprint (or bar code), optical spectroscopy to monitor biomarkers associated with diabetes in the exhaled breath of a user, including but not limited to acetone, methanol, ethanol, carbon monoxide, nitric oxide, low-carbon alkanes, optical spectroscopy cancer spectral fingerprint (or bar code, etc.).

Using optical probe and/or fiber optical probe to detect the biomarkers of AD from user's body fluids and/or tissues: The data detection component(s) 114a-n may comprise an optical component 418. The optical component 418 may be configured to inject incident light and detect the biological and biochemical information of a user. The optical component 418 may be configured to detect the biomarkers of AD in a user's body fluid (including but not limited to blood, serum, saliva, urine, sweat, tears, cerebrospinal fluid (CSF), pleural effusion, subcutaneous body fluids (including but not limited to subcutaneous blood, interstitial fluids, lymph, etc.), tissue fluids, semen, gynecological secretions, etc.) and/or tissues.

The optical component 418 can include spectroscopic probes and/or fiber optical probes (referred to as detection probes) and/or a probe window that the emits light. A fiber optical probe that its end is an optic window can pass deep through a nasal cavity and reach the position near and/or at the hippocampus, amygdala, substantia nigra area, and/or pineal gland in the user's brain with image guidance methods including but not limited to an optical image probe that can be built in the same flexible cable with the optical component 418, or separate optical image probe with flexible fiber, and/or ultrasound imaging technology, etc. to achieve monitoring purpose, perform minimal invasive or noninvasive detection work for data collection. The incident light is incident on a sample that position is near the area of hippocampus, substantia nigra, and/or pineal gland in the user's brain; a fiber optical probe receives the optical signal from the samples. An optic fiber can be physically fixed around the user's nostril area, e.g., by medical tapes, and/or be fixed inside a user's nasal cavity by biochemical method, to perform data collection with wearable device monitoring, or desktop device monitoring. The light signal that can be collected from samples containing user's biological and biochemical information from a user's body fluids and/or tissues, to non-invasively or minimal invasively detect the biomarkers of Alzheimer's disease (AD) in user's body fluids and/or tissues including subcutaneous body fluids and capillaries close to the epidermis; or use a wearable instrument and device to make incident light irradiate on the tears on the surface of the user's eyes to detect the biomarkers of AD in the tears.

After necessary data processing (including but not limited to data fitting, noise reduction, etc.), the client system 111a can wirelessly or by wire upload the detection data to the receiving end, including but not limited to personal mobile phones, computers (including tablets, PADs, etc.), wearable devices wireless display equipment, detection system database center, remote expert system). The detection data can be collected from user A, user B, . . . user N, etc., all those data can be uploaded to the cloud and AI-algorithm-based database to carry out data analysis, to classify AD related user group and health group.

The biomarkers of AD include but not limited to genes and proteins associated with AD (including but not limited to tau protein (including but not limited to total tau (t-tau)), phosphorylated tau (p-tau)181, p-tau217 and p-tau231, etc.), β-amyloid (amyloid β-protein, Aβ, including but not limited to Aβ₄₀, Aβ₄₂, Aβ₁₋₄₀, Aβ₁₋₄₂, etc.), neurofilament light chain (NfL), human body fluid (including but not limited to plasma, urine, CSF, etc.) proteins and/or genes including but not limited to AD7c-NTP, CASP3, CD33, CD84, EIF4B, EIF4EBP1, F2R, FAM3B, FGFBP1, GDF15, GFAP, KLK4, KLK14, LIFR, LTBP2, MAPT, NEFL, NELL1, NUCB2, PARP1, PDGFC, PPY, PRDX5, PSME1, RBKS, TNFSF14 and YKL-40, etc., Lipoprotein E gene, β-secretase 1 (Beta-site app cleaving enzyme 1, BACE1), presenilin (PSEN), microglial receptor (CD33), phosphorylated tau protein, β-synaptic co-Nuclear protein (β-synuclein), Astrocytic α7-Subunit of The Nicotinic Acetylcholine Receptors (α7nAChRs), Melatonin, aluminum-containing biochemicals, etc.

Using optical probe and/or fiber optical probe to detect the biomarkers of PD from user's body fluids and/or tissues: The data detection component(s) 114a-n may comprise an optical component 418. The optical component 418 may be configured to inject incident light and detect the biological and biochemical information of a user. The optical component 418 may be configured to detect the biomarkers of PD in a user's body fluid (including but not limited to blood, serum, saliva, urine, sweat, tears, cerebrospinal fluid (CSF), pleural effusion, subcutaneous body fluids (including but not limited to subcutaneous blood, interstitial fluids, lymph, etc.), tissue fluids, semen, gynecological secretions, etc.), and/or tissues. The optical component 418 can include spectroscopic probes or fiber optical probes (referred to as detection probes) and/or a probe window that the emits light.

A fiber optical probe that its end is an optic window can pass deep through nasal cavity and reach the position near and/or at hippocampus, amygdala, substantia nigra area, and/or pineal gland in the user's brain with image guidance methods including but not limited to an optical image probe that can be built in the same flexible cable with the optical component 418, or separate optical image probe with flexible fiber, and/or ultrasound imaging technology, etc. to achieve monitoring purpose, perform minimal invasive or noninvasive detection work for data collection. The incident light is incident on a sample that position is near and/or at the area of hippocampus, amygdala, substantia nigra area, and/or pineal gland in the user's brain; a fiber optical probe receives the optical signal from the samples. An optic fiber can be physically fixed around the user's nostril area, e.g., by medical tapes, and/or be fixed inside a user's nasal cavity by biochemical method, to perform data collection with wearable device monitoring, or desktop device monitoring. The light signal that can be collected from samples containing user's biological and biochemical information from user's body fluids and/or tissues, to non-invasively or minimal invasively detect the biomarkers of cancers in user's body fluids and/or tissues, including subcutaneous body fluids and capillaries close to the epidermis; or use a wearable instrument and device to make incident light irradiate on the tears on the surface of the user's eyes to detect the biomarkers of PD in the tears. After necessary data processing (including but not limited to data fitting, noise reduction, etc.), the client system 111a can wirelessly or by wire upload the detection data to the receiving end, including but not limited to personal mobile phones, computers (including tablets, PADs, etc.), wearable devices wireless display equipment, detection system database center, remote expert system). The detection data can be collected from user A, user B, . . . user N, etc., all those data can be uploaded to the cloud and AI algorithm based database to carry out data analysis, to classify PD related user group and health group.

The biomarkers of PD include but not limited to genes and proteins associated with PD (including but not limited to α-synuclein, glycoprotein GPNMB, dopamine, including but not limited to cerebrospinal fluid dopamine metabolite DOPAC, and neurofilament light chain Neurofilament light chain (NL), melatonin, abnormal melatonin distribution in the pineal gland, iron-containing compound biochemicals, protein deposition in the substantia nigra, and some of the AD biomarkers listed above.

The optical component 418 can include an optical probe and/or a fiber optical probe with a light source (the light source can be but is not limited to LED, laser, etc.), the laser can be output pulse laser, continuous wave laser (CW Laser). The wavelength of incident light can be in the near-infrared band, visible light band, or near-ultraviolet band. Near-infrared band wavelengths include but are not limited to wavelengths of 1550 nanometers or longer, 1540 nanometers, 1535 nanometers, 1420 nanometers, 1064 nanometers, 830 nanometers, 785 nanometers, etc. Visible light band wavelengths include but are not limited to 633 nanometers, 514 nanometers, and 488 nanometers. etc. UV band wavelengths include but are not limited to 365 nanometers, 355 nanometers, 266 nanometers, 254 nanometers, 248 nanometers, etc. The wavelength of the incident light of the optical probe and/or the fiber optical probe can be adjusted, and the output light of more than one wavelength can be adjusted, and its wavelength includes but is not limited to the above-mentioned light wavelength and at least the second wavelength of the output light is within 20 nanometers; the focal length of the output light of an optical probe is adjustable, adjustment range: 0.01 mm-50 mm. The output power range may be between 0.1 μW (microwatt)-500 mW (milliwatt), such as 0.1 μW, 0.5 μW, 1 μW, 5 μW, 1 μW, 20 μW, 50 μW, 100 μW, 200 μW, 500 μW, 1 mW, 5 mW, 10 mW, 20 mW, 50 mW, 100 mW, 200 mW, 300 mW, 400 mW, etc.

The optical component 418 can receive the optical signal of a sample of a user's body fluids and/or tissues: the incident light can be incident on a sample, and the optical probe can collect and receive the physical, chemical, biological and biochemical, and biochemical information of a sample from or near the sample part resulted from the incident light. The optical signal containing the biological and biochemical information that a device with an optical probe or fiber optical probe collected and received from a user's sample including but not limited to near-infrared absorption signal, infrared absorption signal, far-infrared absorption signal, fluorescence signal, Rayleigh scattering signal, Raman scattering signal, transmission signal, and/or emission signal, etc.

One mode of optical probe and/or fiber optical probe is a fiber optical probe that contains an optical fiber that transmits optical signals for inputting incident light and collecting optical information from samples. The optical fiber probe can be physically flexible and bendable, with length range: 1 cm-200 cm, outer diameter range: 0.5 mm-15 mm (such as 1 mm-10 mm, 1 mm, 2 mm, 5 mm, etc.). The optical fiber probe can pass deep through the nasal cavity and reach the position near and/or at hippocampus, amygdala, substantia nigra area, and/or pineal gland in the brain with image guidance methods including but not limited to an optical image probe that can be built in the same flexible cable with the optical component 418, or separate optical image probe with flexible fiber, and/or ultrasound imaging technology, etc. to achieve monitoring purpose, perform minimal invasive or noninvasive detection work for data collection.

The optical component 418 can use the wearable instrument device mentioned above in this patent to inject incident light on the tear that is on the surface of a user's eye, on a macula (control the wavelength of the incident light (such as wavelength longer than 1.4 μm, near-infrared band wavelength, or select the wavelength of the visible light band under the condition of reducing the incident light power (generally not higher than 20 mW, such as 10 mW, 5 mW, 1 mW, 0.5 mW, 0.2 mW, 0.1 mW, 50 μW, etc.) to achieve eye-safe level), on facial skin (the incident light power is generally not higher than 200 mW, such as 100 mW, 50 mW, 20 mW, 10 mW, 5 mW, 2 mW, 1 mW, etc.), etc., to perform data collection and detection work.

The optical component 418 can integrate optical technology, spectroscopy technology and nanotechnology. The nanotechnology probe module can include a substrate detection probe that includes nano-scale materials on the surface, such as nano-particles of precious metals (including but not limited to silver, gold, copper, platinum, etc., and their alloys and/or oxides), carbon nanotubes, graphene (including but not limited to monolayer graphene, metal compounds containing graphene, graphene related oxides, graphene related alloy, etc.). These substrates can be a silicon chip, a silicon chip with a surface coating of oxide film, and can be made of materials such as glass, metal, aluminum foil, paper, etc. A substrate detection probe can include nano-scale materials on the surface, such as nano-particles, carbon nanotubes, etc., A substrate detection probe can have a shape of chip that surface can be flat, or textured The surface texture can be random, or sub-regular distributed holes or bars that can be made based on self-assembly, self-organized method in the molecule level, the size of those hole and/or bars in X-Y is in the range of 5 nm-1 mm, such as 20 nm-200 nm, the size of those hole and/or bars in Z direction is in the range of 5 nm-1 mm, such as 20 nm-200 nm) etc., filamentous, needle-like, sword-shaped, etc. that the surface (that surface can be flat and/or textured as mentioned above) is with precious metals (including but not limited to silver, gold, copper, platinum, etc., and their alloys and/or oxides, graphene (including but not limited to monolayer graphene, metal compounds containing graphene, including graphene oxides, etc.).

The nanotechnology probe module, can include a liquid or sol detection probe that can have nano particles contained in sol (nanosol), liquid, etc., that includes nano-scale materials such as nano-particles of precious metals (including but not limited to silver, gold, copper, platinum, etc., and their alloys and/or oxides), carbon nanotubes, graphene (including but not limited to monolayer graphene, metal compounds containing graphene, graphene related oxides, graphene related alloy, etc.).

A test sample (the physical form of a sample includes but is not limited to liquid, sol state, human body fluids, human tissues, etc.) can be directly dropped on one of above mentioned a substrate detection probes, and a sensitizer (including but not limited to binary ionic compound aqueous solutions combining elements from the first main group and the seventh main group in The Periodic Table of Elements, including but not limited to NaCl, KI and other aqueous solutions) can be added for detection. Alternatively, the sample can first be added to the liquid or sol detection probe, a sensitizer can be added, and then add drops of the liquid and/or sol containing a sample to be detected on the substrate detection probe. The above-mentioned optical probe and/or fiber optical probe can be used to focus the emitted light on the sample surface, on the interface between the sample and the surface containing nanostructure material, on nanosol containing sample, or on the detection probe containing the detection sample. Optical probes and fiber optical probes receive optical information containing sample's chemical and biochemical information of a user caused by the sample being irradiated by incident light, and then perform sample data collection, storage, and data processing (including but not limited to noise reduction, data fitting based on the standard samples, etc.), and data is uploaded to a server and a cloud platform database management center.

The instruments and devices with optical probe or fiber optical probe applied the optical technology and optical spectroscopic methods (including but not limited to reflection spectroscopy, fluorescence spectroscopy, absorption spectroscopy (including near-infrared absorption spectroscopy, mid-infrared absorption spectroscopy, far-infrared absorption spectroscopy, etc.), scattering spectroscopy (including Rayleigh scattering spectroscopy), Raman scattering spectroscopy, nano-enhanced Raman spectroscopy (or surface-enhanced Raman spectroscopy), etc.), and/or emission spectroscopy, etc.), combined with nanotechnology in this invention can be applied to the detection of major diseases (including but not limited to AD, PD, cancer, diabetes, etc.), especially on-site, rapid, high sensitive, low-cost screening of early stage major diseases.

The client system 111a can be only a detection system terminal. Alternatively, the client system 111a can be a detection system terminal with preliminary computer artificial intelligence (AI) algorithm analysis, classification, pattern recognition and other functions. The client system 111a can be used through wired or wireless information transmission function, wired or wireless transmission of sensing detection data to the connected instruments and devices, including but not limited to mobile phones, tablet computers, laptop computers (Laptop), desktop computers, servers (Server), the above-mentioned items of the present invention instruments and devices, etc., for further computer artificial intelligence algorithm analysis, classification, and pattern recognition. The data collected by the client system 111a can be transmitted wired or wirelessly to the connected instruments and devices through wired or wireless information transmission functions, including but not limited to mobile phones, tablets, laptops (Laptops), desktop computers, servers (Servers). The data collected by the client system 111a can be uploaded to the cloud network 102 for comprehensive computer artificial intelligence algorithm analysis, classification, and pattern recognition. The client system 111a can be remotely controlled and interacting with by the evaluation service 118.

The client system 111a may use any available wireless communication standard to communicate with the cloud network 102, including Global System for Mobile Communications (GSM) such as 4G, 5G, and 6G, Universal Mobile Telecommunications Service (UMTS), and Code Multiple Access (CDMA) for communication. GSM can include General Packet Radio Service (GPRS), Enhanced Data Rates for GSM Evolution (EDGE) and Circuit Switched Data Service (CSD). Universal Mobile Telecommunications Service (UMTS) may include Wideband Code Division Multiple Access (WCDMA), High Speed Packet Access (HSPA), High Speed Downlink Packet Access (HSDPA), Universal Mobile Telecommunications Service Time Division Duplex (UMTS-TDD), and Long Term Evolution (LTE). Code Multiple Access (CDMA) can include CDMA2000 and Ultra Mobile Broadband (UMB); it can also use Beidou satellite navigation system, Starlink satellite network communication system, etc., with the help of 4G, 5G, and 6G GPS systems; it can also use Wi-Fi, NearLink, HDMI, AirPlay, Bluetooth, infrared technology, NFC, DLNA, etc.

In embodiments, the client system 111a can determine a condition (e.g., a medical condition) of the particular user A based on the data stored in the personalized database 413. For example, the client system 111a can determine if the user has an early-stage medical condition, such as early-stage dementia, cancer, etc. The client system 111a can generate a personalized recommendation based on the condition, such as the early-stage medical condition, e.g., sub-health diabetes, etc. The client system 111a can present information indicating the personalized recommendation to the user for managing the condition. The client system 111a can present the information indicating the personalized recommendation to the user for managing the condition via the interface 113a-n of the application 112a. Not only for a individual user A, similarly, the client system 111a can determine a condition (e.g., a medical condition) of the particular user B, user C, . . . user N . . . , all monitored detection data can be uploaded to cloud AI algorithm based database to carry out detection end-user integration covering all users involved, data analysis, pattern recognition, then to tell any significant change in a user's daily behavior and/or biological and biochemical condition, and for all users being monitored to classify health, sub-health, and disease (especially early stage diseases) groups.

In embodiments, the client system 111a can comprise an optical probe based device. The client system 111a can include a laser as a light source. The client system 111a can test a user's body fluids (including but not limited to blood, saliva, urine, sweat, tears, cerebrospinal fluid (CSF), pleural effusion, subcutaneous body fluids (including but not limited to subcutaneous blood, interstitial fluids, lymph, etc.), tissue fluids, semen, or/and gynecological secretions, etc.) and tissues to achieve monitoring purpose, to perform minimal invasive or noninvasive detection work for data collection. If the client system 111a comprises an optical probe based device, the client system 111a can receive the optical signal of samples: the incident light can be incident on a liquid phase sample containing body fluids, on skin, on subcutaneous body fluids under the skin surrounding the area that subject to the incident light. The optical probe can collect and receive a light signal from a sample resulting from the incident light.

In embodiments, the client system 111a can comprise an optical probe. The optical probe can collect and receive a light signal from a sample that contains the physical, chemical, biological and biochemical or/and biochemical information (including but not limited to the biomarkers of user's biological and biochemical information related to diseases including but not limited to dementia (including but not limited to Alzheimer's disease, Parkinson disease, etc.), cancers, diabetes, etc., the biomarkers of diseases (including but not limited to AD, PD, cancers, etc.) include but not limited to those biomarkers that can be referred to related section of this patent specification about the biomarkers of diseases, including AD, PD, cancers, diabetes, etc. The light signal collected and received by the client system 111a from a sample may include, but is not limited to, a near-infrared absorption signal, an infrared absorption signal, a far-infrared absorption signal, a fluorescence signal, a Rayleigh scattering signal, a Raman scattering signal, a transmission signal, and/or emission signal, etc.

In some embodiments, the client system 111a can comprise an optic-fiber-based fiber optical probe with a laser as a light source. The fiber optical probe can contain an optic fiber that transmits optical signals for inputting incident light from a sensing device and collecting optical signal from samples by a sensing device. The fiber optical probe can be physically flexible and/or bendable, with an exemplary length range of 1 cm-200 cm and an exemplary outer diameter range of 0.5 mm-15 mm (such as 1 mm-10 mm, 1 mm, 2 mm, 5 mm, etc.). One end of the fiber optical probe can be connected to a sensing device. The other end of the fiber optical probe can be connected to an optical window facing a sample during data collection. The client system 111a including the fiber optical probe can be a wearable device. If the client system 111a including the fiber optical probe is a wearable device, the user can arrange the sensing device in the user's pocket and/or can physically fix it to user's body, e.g., arm, waist, abdomen, back, etc.

The end of the fiber optical probe that is connected to an optical window can pass deep into a nasal cavity of a user and reach the position near and/or at hippocampus, substantia nigra, and/or pineal gland in the user's brain with image guidance methods including but not limited to an optical image probe that can be built in the same flexible cable with the optical component 418, or separate optical image probe with flexible fiber, and/or ultrasound imaging technology, etc. to achieve monitoring purpose, perform minimal invasive or noninvasive detection work for data collection. The incident light can be incident on a sample position that is near the hippocampus, the substantia nigra, or/and the pineal gland area in the user's brain. A fiber optical probe can receive the optical signal from the samples.

The fiber optical probe can be physically fixed around the user's nostril area, e.g., by medical tapes, or/and be fixed inside a user's nasal cavity in a biochemical manner, to perform data collection. A user can apply the client system 111a to carry out wearable continuous real-time and on-site monitoring, e.g., every 3 to 5 minutes a time, to perform a test one time over a certain period of time, such as hours, days, months, etc. with a detection device with fiber optical probe.

The data collected and/or received by the client system 111a can be transferred wirelessly or by wire to remote AI algorithms-based database(s) to carry out data analysis to determine changes (e.g., sudden or gradual changes) as function of time about user's disease stage from collected data related to user's behavior and corresponding changes in his/her daily life, and/or user's biological and biochemical information. The determined changes can be used to determine a user's body's physical and/or biological and biochemical condition related to a disease, including but not limited to dementia (including but not limited to Alzheimer's disease, Parkinson disease, etc.), cancer, diabetes, etc.

If the client system 111a includes the fiber optical probe, the client system 111a can collect and receive a light signal from a sample containing the physical, chemical, biological and biochemical and/or biochemical information (including but not limited to the biomarkers related to but not limited to dementia (including but not limited to Alzheimer's disease, Parkinson disease, etc.), cancers, diabetes, etc.). The light signal that the device collects and receives from a sample may include a near-infrared absorption signal, a mid-infrared absorption signal, a far-infrared absorption signal, a fluorescence signal, a Rayleigh scattering signal, a Raman scattering signal, a transmission signal, and/or an emission signal, etc.

In some embodiments, the client systems 111a-n can include a combination of wearable devices (e.g., with an eye glass type of sensing device), non-wearable devices configured to carry out real-time and on-site continue sensing, through images, video (moving images), audio sensing to monitor user's behaviors and corresponding changes in his/her daily life. Meanwhile, the client systems 111a-n can include an instrument and device with a fiber optical probe that goes though user's nasal cavity to collect data about user's biological and biochemical information (it can be wearable device way, or desktop device way), devices with optical probes to test user's body fluids and/or tissues to collect data about user's biological and biochemical information, to collect user's personalized data related to diseases including but not limited to dementia (including but not limited to Alzheimer's disease, Parkinson disease, etc.), cancers, diabetes, etc.

In embodiments, the above-disclosed instruments and devices with images, videos and audio sensing detection technology methods and optical spectroscopy detection technology method of the present invention can be combined with artificial intelligence (AI) algorithms configured to perform data analysis. Algorithm method can include Classification, Regression, Clustering, Dimensionality Reduction to carry out pattern recognition, supervised and/or unsupervised large-scale training, modeling, optimization, iteration and/or recursion with machine learning method.

In embodiments, the above-disclosed instruments and devices with images, videos and audio sensing detection technology method and optical spectroscopy detection technology method of the present invention can be combined with virtual reality (VR), augmented reality (AR), and/or mixed reality (MR), which can span multiple sensory modalities, including vision, hearing, touch, somatosensory, taste and smell, etc., to conduct real-time cognitive evaluation, interactive tracking, and real-time evaluation of a user.

In embodiments, the above-disclosed instruments and devices with images, videos and audio sensing detection technology method and optical spectroscopy detection technology method of the present invention can be combined with VR, AR, and/or MR to assist with or lead drug development, for editing items related to the cognitive function assessment of a user, for simulating AD or PD symptoms, and/or identifying disease representation characteristics. Entries can be uploaded to the personalized detection database management center in real time for real-time dynamic monitoring and evaluation, and when necessary, feedback information such as improvement tips, suggestions, instructions, etc. will be provided to the monitored user, then feedback to the drug development team to improve, optimize and upgrade the drugs which perform better result.

In embodiments, the optical spectroscopy detection technology methods described herein, combined with the application of VR, AR, MR and other technologies, an intensive training process procedure for diseases such as AD and PD can be compiled, and users can interact and feedback with this process in real time in his/her daily lives. Interactive tracking, real-time monitoring, learning management, and intensive training can be used to slow down the progression of diseases such as AD and PD, thereby improve quality of user's life, and reduce the burden of patient care and medical care on families and society.

In embodiments, the above-disclosed instrument and device with images, videos and audio sensing detection technology methods and optical spectroscopy detection technology and nano technology methods of the present invention, combined with the application of VR, AR and/or MR, can assist or lead drug development. For patients with diseases such as AD, PD, cancers, diabetes, and general healthy people, a blind test after administration can be conducted for a certain period of time during the drug development process to evaluate and compare the performance differences and differential evaluation levels between the patient group and the healthy group after administration. The evaluation level can be used to evaluate the efficacy of the developed drugs, and then to guide the research, development and application of drugs for AD, PD and other diseases.

In embodiments, one or a combination of more than one method as described in the present invention can be employed. This can involve the detection of significant biomarkers of AD, PD, cancers, diabetes, etc., such as genes, proteins, atomic clusters, molecular clusters in human body fluid and tissues, monitoring behavior and corresponding changes of a user's daily activities, including speech of the user, cognitive tests, epidemiological data of individuals, as well as technologies like VR, AR, MR, etc. This can provides valuable evaluation information for the early screening of major diseases such as AD, PD, diabetes, and cancers.

In embodiments, the above-mentioned sensor sensing and testing terminals can be connected through mobile Internet, and end-customer, integrated interactions can be uploaded to the encrypted cloud platform sensing testing database, remote expert consulting service system, and customer service system management center to perform artificial intelligence algorithms and big data, big model analysis. The techniques described herein can be used to classify disease stages, track medical efficacy, and/or develop new drugs.

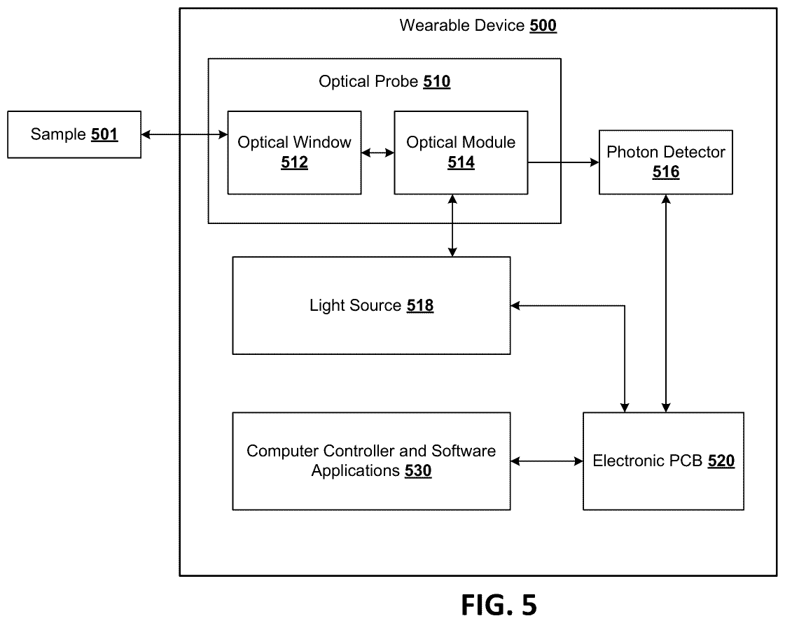

FIG. 5 shows an example wearable device 500 in accordance with the present disclosure. One or more of the client devices 111a-n can be or comprise the wearable device 500. The wearable device 500 can include a light source 518, an optical probe 510, a photo detector 516, computer controller and software application 530, and an electronic printed circuit board (PCB) 520, etc. The optical probe 510 can include an optical window 512 and one or more optical modules 514. The optical probe 510 can include but not limited to spectroscopic probes and/or fiber optical probes (e.g., detection probes). The optical modules 514 can include one or more of mirrors, fibers, lenses, prisms, optical glasses, etc.

The computer controller and software applications 530 can include one or more processors, application software, and/or the like. The computer controller and software applications 530 can include a computer controller. The computer controller can be built into the wearable device 500, or the computer controller can be separate from the wearable device 500 and in communication with the wearable device 500 via one or more wired or wireless connections. The computer controller and software application 530 can control the light source 518 to incident light through the optical probe 510 on a sample 501. For example, the electronic PCB 520 controlled by the computer controller and software applications 530 can execute commands to control the light source 518 to incident light through the optical probe 510 on the sample 501. The light source 518 can include a laser light source. The sample 501 can include, but is not limited to, a user's body fluids and/or tissues. The collected light signal can be associated with a certain body part of a user's body, including but not limited to the user's fingers, wrists, forearms, arms, legs, feet, waist and abdomen, trunk, neck, deep inside nasal cavity, etc., to non-invasively or minimal invasively detect a user's biological and biochemical information at the surface and/or in subcutaneous body fluids and capillaries close to the epidermis at the user body area that closes or even touches the optical window 512.

The optical probe 510 can receive the collected light signal from the sample 501. The photo detector 516 can receive the collected light signal from the optical probe 510. The photo detector 516 can include a charge-coupled device (CCD) or a complementary metal oxide semiconductor (CMOS) sensor to receive the collected light signal from the optical probe 510. The photo detector 516 can convert light photos to photo-electrons. The electronic PCB 520 controlled by the computer controller and software applications 530 can execute commands to support photo detector readings by the photo detector 516.

The computer controller and software applications 530 can analyze the photo detector reading. The computer controller and software applications 530 can analyze the photo detector reading to generate a spectrum analyzed and/or report associated with the sample 501. The computer controller and software applications 530 can send data indicative of the analysis, such as to the evaluation service 118. The computer controller and software applications 530 can send the data indicative of the analysis via wired and/or wireless communication. The data sent by the computer controller and software applications 530 can be stored in one of the personalized databases 122a-n corresponding to the user associated with the wearable device 500.

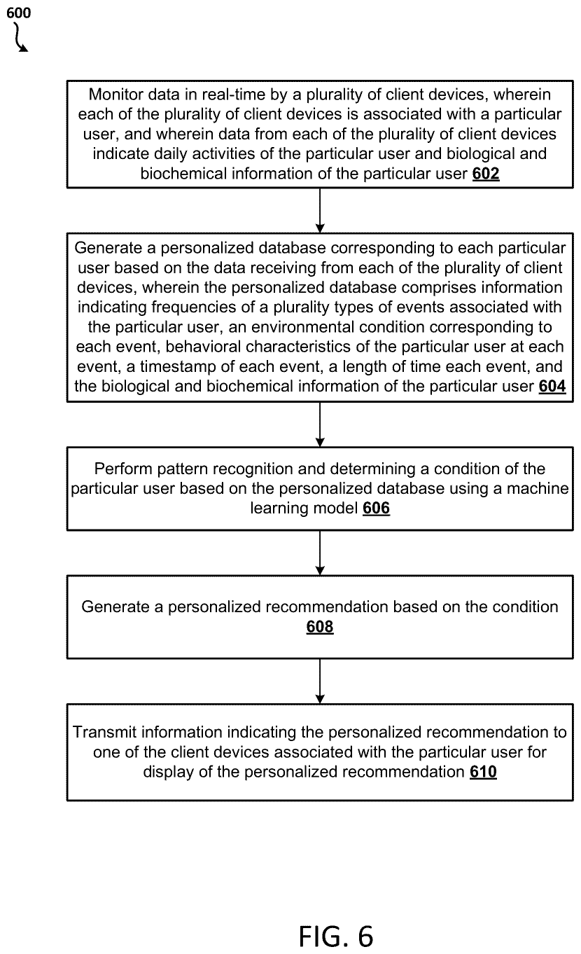

FIG. 6 shows an example server-side process 600 for implementing real-time monitoring and evaluation in accordance with the present disclosure. Although depicted as a sequence of operations in FIG. 6, those of ordinary skill in the art will appreciate that various embodiments may add, remove, reorder, or modify the depicted operations.

At 602, data can be collected and/or received. The data can be collected from a plurality of client systems. The plurality of client systems can monitor the data in real time. Each of the plurality of client systems can be associated with a particular user. Data from each of the plurality of client systems can indicate the behaviors and corresponding changes in the daily activities of the particular user and biological and biochemical information of the particular user. The data can be collected and/or received in real time (e.g., as it is collected by the plurality of client systems).

At 604, a personalized database corresponding to each particular user can be generated. The personalized database corresponding to each particular user can be generated based on the data received from each of the plurality of client systems. The personalized database corresponding to a particular user may store information indicating frequencies of a plurality types of events associated with the particular user. The personalized database corresponding to a particular user may store information indicating an environmental condition corresponding to each event. The personalized database corresponding to a particular user may store information indicating behavioral characteristics of the particular user at each event. The personalized database corresponding to a particular user may store information indicating a timestamp of each event. The personalized database corresponding to a particular user may store information indicating a length of time each event.

At 606, pattern recognition can be performed. A condition of the particular user can be determined based on the personalized database. The pattern recognition can be performed using at least one machine learning model. The condition of the particular user can be determined using at least one machine learning model. At 608, a personalized recommendation can be generated. The personalized recommendation can be generated based on the condition. At 610, information indicating the personalized recommendation can be transmitted (e.g., sent). The information indicating the personalized recommendation can be transmitted to one of the client systems associated with the particular user for display of the personalized recommendation.

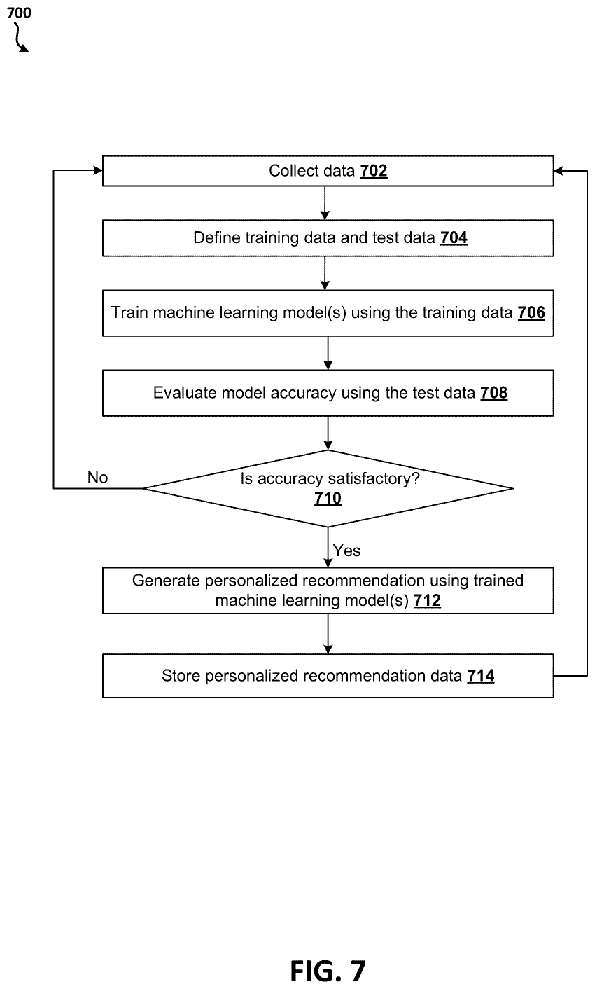

FIG. 7 is a flowchart illustrating an example process 700 for training a machine learning model. A server or other computing device may be used singly or in combination to implement the process 700. Although depicted as a sequence of operations, those of ordinary skill in the art will appreciate that various embodiments may add, remove, reorder, or modify the depicted operations.

At 702, data can be collected. The data can be collected from a plurality of client systems. The plurality of client systems can monitor the data in real time. Each of the plurality of client systems can be associated with a particular user. Data from each of the plurality of client systems can indicate a user's behavior and corresponding changes in his or her daily activities of the particular user and biological and biochemical information of the particular user. The data can be collected and/or received in real time (e.g., as it is collected by the plurality of client systems).

At 704, training data and test data are defined. Preferably, two separate and independent sets of data are defined as training data and test data, respectively. The set of training data will be used to train machine learning models for condition prediction. The set of test data will be used to test the machine learning models and evaluate their condition prediction accuracy. At 706, machine learning models can be trained using the set of training data. To well the machine learning models, it is preferable to provide sufficient training data. At 708, the performance of the trained machine learning models can be evaluated. The performance of the trained machine learning models can be evaluated using the set of test data. Any suitable techniques may be employed to determine the accuracy of evaluation.

At 710, it can be determined whether the condition prediction accuracy is satisfactory. In some embodiments, ground truth condition prediction data (e.g., indicated by the test data set) for a particular user can be compared with condition predictions generated by the machine learning models to determine whether the condition prediction accuracy is satisfactory. If the condition prediction is satisfactory, the process 700 may proceed to 712. At 712, the trained machine learning models may be used to generate personalized recommendations. At 714, the personalized recommendations can be stored. On the other hand, if the condition prediction accuracy is not satisfactory, the process 700 may return to operation 702 and the operations 702 through 710 can be iterated using any suitable model iteration methods, including, but are not limited to, online incremental learning methods (e.g., directly applying newly added data to further update the original model, the model is learned online, and machine learning obtains an original algorithm model that further amplifies the new data), methods of merging data (e.g., merging the algorithm training data that has been completed in stages and the newly added data, the model learns all the data offline, and machine learning obtains a new algorithm model), model fusion methods (e.g., using the prediction results of the original model as newly added features, and training a new algorithm model on the new data), and/or the like. It should be appreciated that any suitable techniques for determining condition prediction accuracy may be employed.

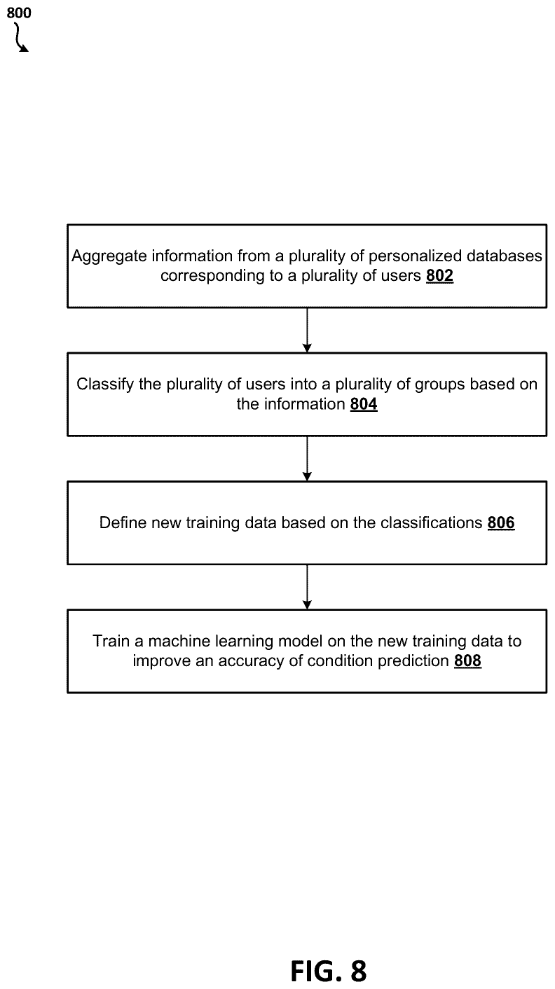

FIG. 8 shows an example process 800 for training a machine learning model in accordance with the present disclosure. Although depicted as a sequence of operations in FIG. 8, those of ordinary skill in the art will appreciate that various embodiments may add, remove, reorder, or modify the depicted operations.

A training database can include (e.g., store) training data for training one or more machine learning models. To generate the training data, information can be aggregated. At 802, information can be aggregated from a plurality of personalized databases. Each of the plurality of personalized databases can correspond to a particular user from a plurality of users. The personalized database corresponding to each particular user can be generated based on the data received from each of the plurality of client systems. The personalized database corresponding to a particular user may store information indicating frequencies of a plurality types of events associated with the particular user. The personalized database corresponding to a particular user may store information indicating an environmental condition corresponding to each event. The personalized database corresponding to a particular user may store information indicating behavioral characteristics of the particular user at each event. The personalized database corresponding to a particular user may store information indicating a timestamp of each event. The personalized database corresponding to a particular user may store information indicating and a length of time each event.

At 804, the plurality of users can be classified. The plurality of users can be classified into a plurality of groups. The plurality of users can be classified into the plurality of groups based on the aggregated information. At 806, new training data can be defined. The new training data can be defined based on the classifications (e.g., the classifications of the plurality of users into the plurality of groups). The new training data can be stored, such as in a training database. At 808, the machine learning model(s) can be trained on the new training data. Training the machine learning model(s) on the new training data can improve an accuracy of condition prediction.

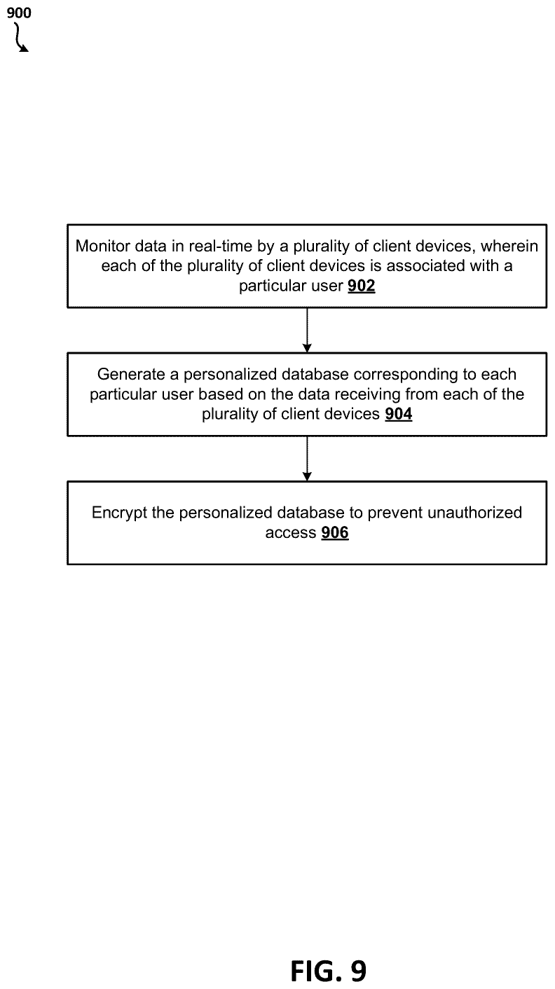

FIG. 9 shows an example process 900 for encrypting a database that is local or cloud based in accordance with the present disclosure. Although depicted as a sequence of operations in FIG. 9, those of ordinary skill in the art will appreciate that various embodiments may add, remove, reorder, or modify the depicted operations.

At 902, data can be collected and/or received. The data can be collected from a plurality of client systems. The plurality of client systems can monitor the data in real time. Each of the plurality of client systems can be associated with a particular user. Data from each of the plurality of client systems can indicate the behaviors and corresponding changes in the daily activities of the particular user and biological and biochemical information of the particular user. The data can be collected and/or received in real time (e.g., as it is collected by the plurality of client systems).

At 904, a personalized database corresponding to each particular user can be generated. The personalized database corresponding to each particular user can be generated based on the data received from each of the plurality of client systems. The personalized database corresponding to a particular user may store information indicating frequencies of a plurality types of events associated with the particular user. The personalized database corresponding to a particular user may store information indicating an environmental condition corresponding to each event. The personalized database corresponding to a particular user may store information indicating behavioral characteristics of the particular user at each event. The personalized database corresponding to a particular user may store information indicating a timestamp of each event. The personalized database corresponding to a particular user may store biological and biochemical information about the user. The personalized database corresponding to a particular user may store information indicating and a length of time each event. At 906, the personalized database can be encrypted. The personalized database can be encrypted to prevent unauthorized access to the data contained (e.g., stored) in the personalized database.

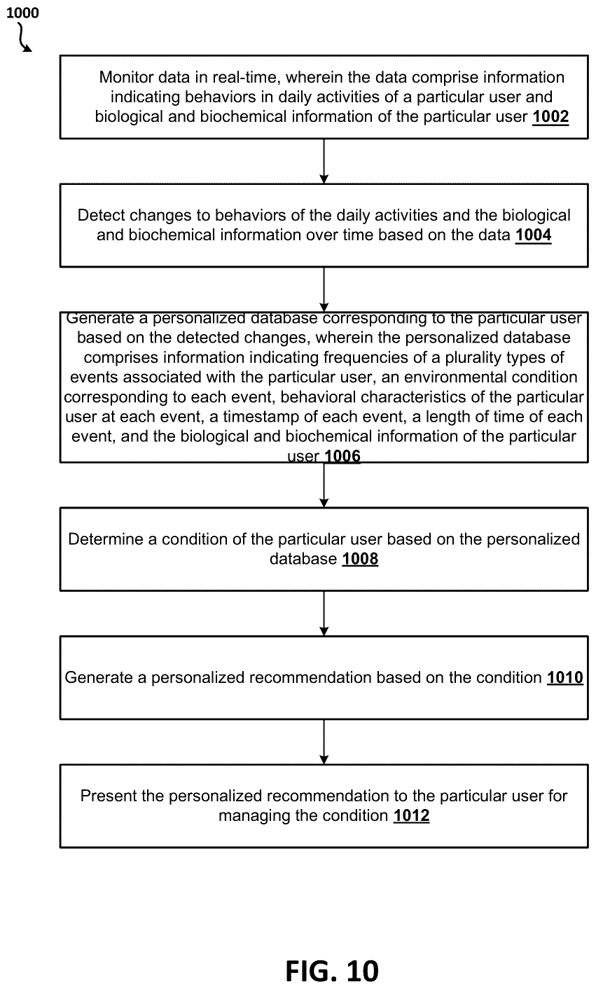

FIG. 10 shows an example client-side process 1000 for implementing real-time monitoring and evaluation in accordance with the present disclosure. Although depicted as a sequence of operations in FIG. 10, those of ordinary skill in the art will appreciate that various embodiments may add, remove, reorder, or modify the depicted operations.

At 1002, data can be collected and/or monitored in real time. The data can be collected and/or monitored in real time by client system. The data collected and/or monitored may comprise information indicating the behaviors and corresponding changes of daily activities of a user of that client system and/or biological and biochemical information of the user. The client systems may comprise one or more data detection components. The data detection component(s) may be components (e.g., sensors, etc.) configured to collect and/or monitor the data, such as in real-time. The data detection component(s) may comprise a visual component, a hearing component, and/or an optical component.

At 1004, changes to the behaviors in the daily activities of the user and/or changes to the biological and biochemical information of the user can be detected. The changes to the behaviors in the daily activities of the user and/or changes to the biological and biochemical information of the user can be detected based on (e.g., using) the data. At 1006, a personalized database can be generated. The personalized database can be generated based on the detected changes. The personalized database can be stored locally (e.g., on the client system). The personalized database can be stored remotely on AI algorithm based cloud platform. The personalized database can include information indicating frequencies of a plurality types of events associated with the particular user. The personalized database can include information indicating an environmental condition corresponding to each event. The personalized database can include information indicating behavioral characteristics and corresponding changes of the particular user at each event. The personalized database can include information indicating a timestamp of each event. The personalized database can include information indicating a length of time of each event. The personalized database can include information indicating the stage and changes of biological and biochemical information of the user.

At 1008, a condition can be determined. The condition can be, for example, a medical condition of the user. The condition can be determined based on the data stored in the personalized database. For example, it can be determined if the user has an early-stage medical condition, such as early-stage dementia, cancers, etc. At 1010, a personalized recommendation can be generated. The personalized recommendation can be generated based on the condition, such as the early-stage medical condition. At 1012, information indicating the personalized recommendation can be presented. The information indicating the personalized recommendation can be presented to the user (e.g., via an interface of the client system) for managing the condition.

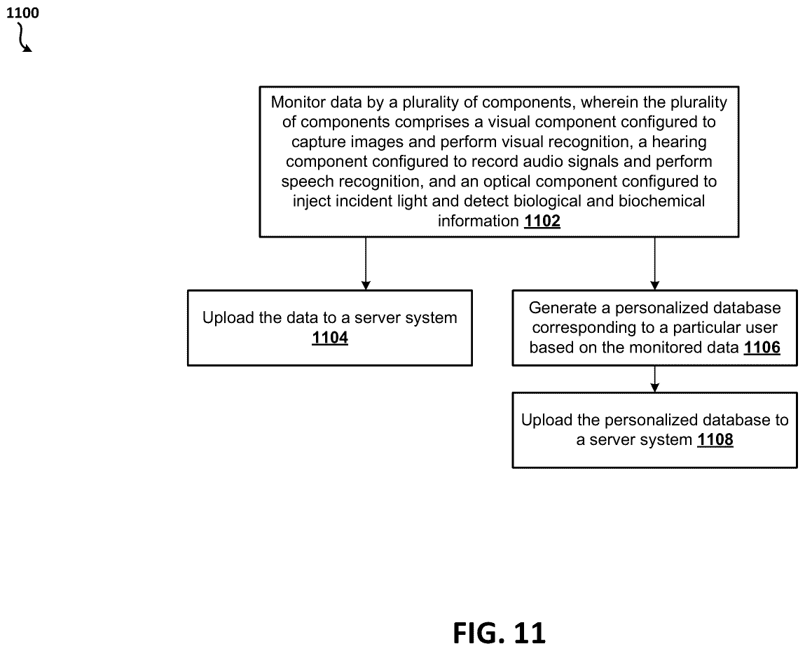

FIG. 11 shows an example process 1100 for uploading data in accordance with the present disclosure. Although depicted as a sequence of operations in FIG. 11, those of ordinary skill in the art will appreciate that various embodiments may add, remove, reorder, or modify the depicted operations.

At 1102, data can be collected and/or monitored in real time. The data can be collected and/or monitored in real time by client system. The data collected and/or monitored may comprise information indicating the behavior and corresponding changes of daily activities of a user of that client system and/or biological and biochemical information of the user. The client system may comprise one or more data detection components. The data detection component(s) may be components (e.g., sensors, etc.) configured to collect and/or monitor the data, such as in real-time. The data detection component(s) may comprise a visual component. The visual component may be configured to capture images and perform visual recognition. The data detection component(s) may comprise a hearing component. The hearing component may be configured to record audio signals and perform speech recognition. The data detection component(s) may comprise an optical component. The optical component may be configured to inject incident light and detect the biological and biochemical information of a user.

At 1104, the data can be uploaded. The data can be uploaded to a server system, such as a cloud server system or a local server system. Alternatively, the data can be used to generate a personalized database. At 1106, a personalized database can be generated based on the monitored data. The personalized database can correspond to the user. The personalized database can be stored locally (e.g., on the client system). The personalized database can include information indicating frequencies of a plurality types of events associated with the particular user. The personalized database can include information indicating an environmental condition corresponding to each event. The personalized database can include information indicating behavioral characteristics and corresponding changes of the particular user at each event. The personalized database can include information indicating a timestamp of each event. The personalized database can include information indicating a length of time of each event. The personalized database can include information indicating the stage and changes of biological and biochemical information of a user. At 1108, the personalized database can be uploaded to a server system, such as a cloud server system, or a local server system.

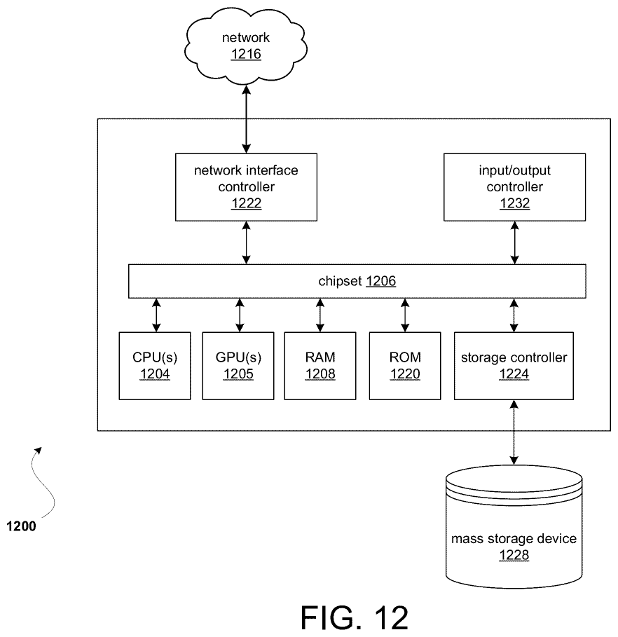

FIG. 12 illustrates a computing device that may be used in various aspects, such as the models, components, and/or devices depicted in any of FIGS. 1-5. With regard to FIGS. 1-4, any or all of the components may each be implemented by one or more instance of a computing device 1200 of FIG. 12. The computer architecture shown in FIG. 12 shows a conventional server computer, workstation, desktop computer, laptop, tablet, network appliance, PDA, e-reader, digital cellular phone, or other computing node, and may be utilized to execute any aspects of the computers described herein, such as to implement the methods described herein, such as the methods shown in FIGS. 6-11.

The computing device 1200 may include a baseboard, or “motherboard,” which is a printed circuit board (PCB) to which a multitude of components or devices may be connected by way of a system bus or other electrical communication paths. One or more central processing units (CPUs) 1204 may operate in conjunction with a chipset 1206. The CPU(s) 1204 may be augmented with or replaced by other processing units, such as GPU(s) 1205 that can be specialized for but not necessarily limited to highly parallel computations, such as graphics and other visualization-related processing.

A chipset 1206 may provide an interface between the CPU(s) 1204 and the remainder of the components and devices on the baseboard. The chipset 1206 may provide an interface to a random-access memory (RAM) 1208 used as the main memory in the computing device 1200. The chipset 1206 may further provide an interface to a computer-readable storage medium, such as a read-only memory (ROM) 1220 or non-volatile RAM (NVRAM) (not shown), for storing basic routines that may help to start up the computing device 1200 and to transfer information between the various components and devices. ROM 1220 or NVRAM may also store other software components necessary for the operation of the computing device 1200 in accordance with the aspects described herein.

The computing device 1200 may operate in a networked environment using logical connections to remote computing nodes and computer systems through local area network (LAN). The chipset 1206 may include functionality for providing network connectivity through a network interface controller (NIC) 1222, such as a gigabit Ethernet adapter. A NIC 1222 may be capable of connecting the computing device 1200 to other computing nodes over a network 1212.

The computing device 1200 may be connected to a mass storage device 1228 that provides non-volatile storage for the computer. The mass storage device 1228 may store system programs, application programs, other program modules, and data, which have been described in greater detail herein. The mass storage device 1228 may be connected to the computing device 1200 through a storage controller 1224 connected to the chipset 1206. The mass storage device 1228 may consist of one or more physical storage units. The mass storage device 1228 may store an operating system utilized to control the operation of the computing device 1200. The mass storage device 1228 may comprise a management component 1210.

By way of example and not limitation, computer-readable storage media may include volatile and non-volatile, transitory computer-readable storage media and non-transitory computer-readable storage media, and removable and non-removable media implemented in any method or technology.

As will be appreciated by one skilled in the art, the methods and systems may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium may be utilized including hard disks, CD-ROMs, optical storage devices, or magnetic storage devices.

The various features and processes described above may be used independently of one another or may be combined in various ways. All possible combinations and sub-combinations are intended to fall within the scope of this disclosure. In addition, certain methods or process blocks may be omitted in some implementations. The methods and processes described herein are also not limited to any particular sequence, and the blocks or states relating thereto may be performed in other sequences that are appropriate. For example, described blocks or states may be performed in an order other than that specifically described, or multiple blocks or states may be combined in a single block or state. The example blocks or states may be performed in serial, in parallel, or in some other manner. Blocks or states may be added to or removed from the described example embodiments. The example systems and components described herein may be configured differently than described. For example, elements may be added to, removed from, or rearranged compared to the described example embodiments.

It will also be appreciated that various items are illustrated as being stored in memory or on storage while being used, and that these items or portions thereof may be transferred between memory and other storage devices for purposes of memory management and data integrity. Alternatively, in other embodiments, some or all of the software modules and/or systems may execute in memory on another device and communicate with the illustrated computing systems via inter-computer communication. Furthermore, in some embodiments, some or all of the systems and/or modules may be implemented or provided in other ways, such as at least partially in firmware and/or hardware, including, but not limited to, one or more application-specific integrated circuits (“ASICs”), standard integrated circuits, controllers (e.g., by executing appropriate instructions, and including microcontrollers and/or embedded controllers), field-programmable gate arrays (“FPGAs”), complex programmable logic devices (“CPLDs”), etc. Some or all of the modules, systems, and data structures may also be stored (e.g., as software instructions or structured data) on a computer-readable medium, such as a hard disk, a memory, a network, or a portable media article to be read by an appropriate device or via an appropriate connection. The systems, modules, and data structures may also be transmitted as generated data signals (e.g., as part of a carrier wave or other analog or digital propagated signal) on a variety of computer-readable transmission media, including wireless-based and wired/cable-based media, and may take a variety of forms (e.g., as part of a single or multiplexed analog signal, or as multiple discrete digital packets or frames). Such computer program products may also take other forms in other embodiments. Accordingly, the present invention may be practiced with other computer system configurations.

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its operations be performed in a specific order. Accordingly, where a method claim does not actually recite an order to be followed by its operations or it is not otherwise specifically stated in the claims or descriptions that the operations are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

It will be apparent to those skilled in the art that various modifications and variations may be made without departing from the scope or spirit of the present disclosure. Other embodiments will be apparent to those skilled in the art from consideration of the specification and practices described herein. It is intended that the specification and example figures be considered as exemplary only, with a true scope and spirit being indicated by the following claims.

What is claimed is:

1. A system for implementing real-time monitoring and evaluation, comprising:
at least one processor; and
at least one memory communicatively coupled to the at least one processor and comprising computer-readable instructions that upon execution by the at least one processor cause the at least one processor to perform operations comprising:
monitoring data in real-time by a plurality of client systems, wherein each of the plurality of client systems is associated with a particular user, and wherein data from each of the plurality of client systems indicate the behaviors and corresponding changes in the daily activities of the particular user and biological and biochemical information of the particular user;
generating a personalized database corresponding to each particular user based on the data received from each of the plurality of client systems, wherein the personalized database comprises information indicating frequencies of a plurality types of events associated with the particular user, an environmental condition corresponding to each event, behavioral characteristics and corresponding changes of the particular user at each event, a timestamp of each event, a length of time each event, and the biological and biochemical information of the particular user;
performing pattern recognition and determining a condition of the particular user based on the personalized database using a machine learning model;
generating a personalized recommendation based on the condition; and
transmitting information indicating the personalized recommendation to one of the client systems associated with the particular user for display of the personalized recommendation.

2. The system of claim 1, wherein the data from each of the plurality of client systems are detected by a plurality of components, wherein the plurality of components comprises a visual component configured to capture images and videos and perform visual recognition, a hearing component configured to record audio signals and perform speech recognition, and an optical component configured to inject incident light and detect the biological and biochemical information of the particular user.

3. The system of claim 1, the operations further comprising:
aggregating information from a plurality of personalized databases corresponding to a plurality of users; and
classifying the plurality of users into a plurality of groups based on the information.

4. The system of claim 3, the operations further comprising:
defining new training data; and
training the machine learning model on the new training data to improve an accuracy of condition prediction.

5. The system of claim 1, wherein the condition comprises an early-stage dementia.

6. The system of claim 1, the operations further comprising:
comparing data stored in the personalized databases corresponding to each particular user to identify a change and/or a cause of each event.

7. A method for implementing real-time monitoring and evaluation, comprising:
monitoring data in real-time by a plurality of client systems, wherein each of the plurality of client systems is associated with a particular user, and wherein data from each of the plurality of client systems indicate the behaviors and corresponding changes in daily activities of the particular user and biological and biochemical information of the particular user;
generating a personalized database corresponding to each particular user based on the data receiving from each of the plurality of client systems, wherein the personalized database comprises information indicating frequencies of a plurality types of events associated with the particular user, an environmental condition corresponding to each event, behavioral characteristics and corresponding changes of the particular user at each event, a timestamp of each event, a length of time each event, and the biological and biochemical information of the particular user;
performing pattern recognition and determining a condition of the particular user based on the personalized database using a machine learning model;
generating a personalized recommendation based on the condition; and
transmitting information indicating the personalized recommendation to one of the client systems associated with the particular user for display of the personalized recommendation.

8. The method of claim 7, wherein the data from each of the plurality of client systems are detected by a plurality of components, wherein the plurality of components comprises a visual component configured to capture images and videos and perform visual recognition, a hearing component configured to record audio signals and perform speech recognition, and an optical component configured to inject incident light and detect the biological and biochemical information of the particular user.

9. The method of claim 7, further comprising:
aggregating information from a plurality of personalized databases corresponding to a plurality of users; and
classifying the plurality of users into a plurality of groups based on the information.

10. The method of claim 9, further comprising:
defining new training data; and
re-training the machine learning model on the new training data through an iterative process to improve an accuracy of condition prediction.

11. The method of claim 7, wherein the condition comprises an early-stage dementia.

12. A system for implementing real-time monitoring and evaluation, comprising:
at least one processor; and
at least one memory communicatively coupled to the at least one processor and comprising computer-readable instructions that upon execution by the at least one processor cause the at least one processor to perform operations comprising:
monitoring data in real-time, wherein the data comprise information indicating the behaviors and corresponding changes in the daily activities of a particular user and biological and biochemical information of the particular user;
detecting changes of behavior of the daily activities and the biological and biochemical information of the particular user over time based on the data;
generating a personalized database corresponding to the particular user based on the detected changes, wherein the personalized database comprises information indicating frequencies of a plurality types of events associated with the particular user, an environmental condition corresponding to each event, behavioral characteristics and corresponding changes of the particular user at each event, a timestamp of each event, a length of time of each event, and the biological and biochemical information of the particular user;
determining a condition of the particular user based on the personalized database;
generating a personalized recommendation based on the condition; and
presenting the personalized recommendation to the particular user for managing the condition.

13. The system of claim 12, the operations further comprising:
monitoring the data by a plurality of components, wherein the plurality of components comprises a visual component configured to capture images and videos and perform visual recognition, a hearing component configured to record audio signals and perform speech recognition, and an optical component configured to inject incident light and detect the biological and biochemical information of the particular user.

14. The system of claim 12, the operations further comprising:
uploading the data to a server system; or
uploading the personalized database to the server system.

15. The system of claim 12, the operations further comprising:
monitoring the data in real-time by a wearable device.

16. The system of claim 12, the operations further comprising:
monitoring the data by a device comprising an optical probe, wherein the device is configured to collect the biological and biochemical information based on receiving a light signal from a biological and biochemical sample of the particular user.

17. The system of claim 16, wherein the optical probe comprises an optic fiber configured to pass deep through a nasal cavity to collect the biological and biochemical information of the particular user.

18. The system of claim 12, the operations further comprising:
comparing data stored in the personalized databases corresponding to each particular user to identify a change and/or a cause of each event.
